(19) United States
(12) Patent Application Publication
(10) Pub. No.: US 2020/0088615 A1
(43) Pub. Date: Mar. 19, 2020

(54) SYSTEMS AND METHODS FOR RAPID DETECTION OF AN ANALYTE OF INTEREST

(71) Applicant: 3M INNOVATIVE PROPERTIES COMPANY, St. Paul, MN (US)

(72) Inventors: Raj Rajagopal, Woodbury, MN (US); Evan D. Brutinel, Inver Grove Heights, MN (US); Ramasubramani Kuduva Raman Thanumoorthy, Woodbury, MN (US)

(21) Appl. No.: 16/467,658

(22) PCT Filed: Nov. 29, 2017
(86) PCT No.: PCT/US2017/063564

§ 371 Date: Jun. 7, 2019

Related U.S. Application Data
(60) Provisional application No. 62/432,367, filed on Dec. 9, 2016.

(51) Int. Cl. G01N 1/40; B01L 3/00; G01N 33/00

(52) U.S. Cl. CPC G01N 1/4077; B01L 3/5021; G01N 33/0044; B01L 2300/042; G01N 2001/4083; B01L 2300/0832; B01L 2300/0851; B01L 2300/0893; B01L 2300/161; B01L 2300/044

(57) ABSTRACT

Systems and methods for detecting an analyte of interest. The method can include providing a container (102) adapted to receive a sample (152). The container can include a microstructured surface (130). The method can further include positioning a sample in the container; adding an H2S probe and an enzyme substrate to the container; centrifuging the container toward the microstructured surface to form a sediment and a supernatant of the sample; inverting the container, after centrifuging the container, to remove at least a portion of the supernatant from being in contact with the microstructured surface; and interrogating the concentrate in the microstructured surface for the analyte of interest.

### FIELD

The present disclosure generally relates to methods for detecting an analyte of interest, such as bacteria, in a sample, and particularly, to rapid detection of an analyte of interest in a relatively large sample volume.

### BACKGROUND

Testing aqueous samples for the presence of microorganisms (e.g., bacteria, viruses, fungi, spores, etc.) and/or other analytes of interest (e.g., toxins, allergens, hormones, etc.) can be important in a variety of applications, including food and water safety, infectious disease diagnostics, and environmental surveillance. For example, anaerobic or recirculating water used in the oil and gas industry may contain or acquire microorganisms or other analytes, such as sulfate reducing bacteria (SRB), which can flourish or grow as a function of the environment in which they are located. SRB are ubiquitous in seawater, surface water that contains decaying organic matter, and in sediments found in marine and freshwater environments. SRB are commonly found in anaerobic environments, although it has been reported that at least some SRB may tolerate and reproduce in environments that have at least low levels of oxygen.

The growth of SRB may have detrimental effect on industrial processes, for example, causing microbial induced corrosion. SRB obtain energy by oxidizing organic compounds or molecular hydrogen. They use sulfate as an electron acceptor to produce hydrogen sulfide (H₂S). Hydrogen sulfide production can contribute to corrosion of metals (e.g., metals that are used to produce pipes). This corrosion can result in disintegration of the metal and, ultimately, increased maintenance or failure of metal pipes. Biogenic sulfide can also cause corrosion of other materials such as concrete.

By way of further example, a variety of analytical methods can be performed on samples of industrial samples (e.g., groundwater, recirculating water used in the oil and gas industry, cooling towers, etc.) to determine if a sample contains a particular analyte. For example, recirculating water used in the oil and gas industry and cooling tower water can be tested for a microorganism or a chemical toxin. However, there remains a need for improved methods for the detection of SRB.

### SUMMARY

Some aspects of the present disclosure provide a method of detecting an analyte of interest. The method include providing a container adapted to receive a sample, the container comprising a microstructured surface; positioning a sample in the container; adding an H₂S probe and an enzyme substrate to the container; centrifuging the container toward the microstructured surface to form a sediment and a supernatant of the sample; inverting the container, after centrifuging the container, to remove at least a portion of the supernatant from being in contact with the microstructured surface, such that a concentrate of the sample is retained in the microstructured surface, the concentrate comprising the sediment; and interrogating the concentrate in the microstructured surface for the analyte of interest.

Some aspects of the present disclosure provide a method of detecting an analyte of interest. The method include providing a container adapted to receive a sample, the container having an H₂S probe and an enzyme substrate, wherein the container comprises a microstructured surface; positioning a sample in the container; centrifuging the container toward the microstructured surface to form a sediment and a supernatant of the sample; inverting the container, after centrifuging the container, to remove at least a portion of the supernatant from being in contact with the microstructured surface, such that a concentrate of the sample is retained in the microstructured surface, the concentrate comprising the sediment; and interrogating the concentrate in the microstructured surface for the analyte of interest.

Some aspects of the present disclosure provide an article. The article includes a container adapted to receive a sample, the container comprising an open end configured to receive a sample and a closed end, the closed end including a first side comprising a microstructured surface, the first side facing an interior of the container, and a second side opposite the first side and facing outside of the container, wherein at least a portion of the container is substantially transparent such that the microstructured surface is visible from the second side; a probe and an enzyme substrate disposed in the container.

Other features and aspects of the present disclosure will become apparent by consideration of the detailed description and accompanying drawings.

### BRIEF DESCRIPTION OF THE DRAWINGS

FIGS. 1A-1C are side cross-sectional views of a sample detection system according to one embodiment of the present disclosure, which can be used in detecting the presence of an analyte of interest in a sample and illustrate an sample detection method according to one embodiment of the present disclosure.

FIG. 2 is an enlarged schematic partial cross-sectional view of a portion of the sample detection system of FIG. 1 at a point in time.

FIGS. 3A-3D are optical micrographs of the microstructured surface according to one embodiment of the present disclosure.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the term “coupled” and variations thereof are used broadly and encompass both direct and indirect couplings. It is to be understood that other embodiments may be utilized, and structural or logical changes may be made without departing from the scope of the present disclosure. Furthermore, terms such as “top,” “bottom,” and the like are only used to describe elements as they relate to one another, but need not recite specific orientations of the apparatus, to indicate or imply necessary or required orientations of the apparatus, or to specify how the invention described herein will be used, mounted, displayed, or positioned in use.

In a variety of samples that are desired to be tested for an analyte of interest, for example, SRB, a diverse group of microorganisms which grow by coupling the reduction of sulfate (SO₄⁻²) to hydrogen sulfide (H₂S), with the oxidation of non-fermentable organic carbon sources (lactate, acetate, butyrate, etc.). The copious amounts of hydrogen sulfide produced by SRBs have a 3-fold detrimental effect on industrial processes. First, hydrogen sulfide is a major driver of microbiologically influenced corrosion (MIC) causing billions of dollars in damage every year. Second, hydrogen sulfide is a pressing environment, health & safety (EH&S) concern as it is heavier than air, very poisonous, corrosive, flammable, and explosive. Concentrations of hydrogen sulfide in the air over holding ponds can quickly reach levels unsafe for human activity. Third, hydrogen sulfide is an undesired contaminant in oil and gas, lowering the value of products.

In some existing systems and methods to test for SRBs, organisms are inoculated into a medium selective for SRB and hydrogen sulfide is detected by reaction with iron in the medium to form iron sulfide, a black precipitate. Generally, bottles are examined after a 28 day incubation giving the user an order of magnitude estimation of SRB concentration in the original sample. More recently, “time-to-detection” style tests have become available which rely on counting the number of days until the test turns black, as an estimation of initial bacterial number. However, the time-to-result for tests of this type is still 7 days.

The present disclosure generally relates to systems and methods for detecting the presence or absence of (and/or enumerating) an analyte of interest in a sample. Furthermore, the present disclosure generally relates to systems and methods for rapidly detecting the analyte. In some embodiments, the analyte is selected for detecting (e.g., the presence or absence of) sulfate reducing bacteria (SRB). Detection of microorganisms (or other analytes) of interest in a water sample can be difficult, because of the low concentration of these microorganisms. As a result of the low concentration, detection in existing systems and methods can be very slow, because the microorganism(s) need to be grown (or the analyte concentration needs to be increased) to a detectable level, which can take time.

The present inventors, however, have invented systems and methods for greatly decreasing the time needed to detect an analyte of interest in a sample, such as a water sample (e.g. an oil-field or gas-field water sample). The analyte of interest can be analyzed in a test sample that may be derived from any source, such as a sample containing marine water, surface water (e.g., from ponds, lakes or rivers), or sediment from marine or freshwater-sources. In addition, the test sample may be obtained from oil deposits, oil wells, pipelines used to transport oil, or vessels used for storing oil. Particularly, the systems and methods of the present disclosure can include concentrating a sample (e.g., based on density) into a microstructured surface comprising microstructured recesses or wells, wherein each microstructured recess can serve as an individual “test tube” of a small volume (e.g., on the scale of microliters or nanoliters), resulting in a high concentration of the analyte(s) of interest, if present, in the sample. This increase in concentration of the analyte(s) of interest can facilitate and expedite detection of the anlayte(s), for example, for detecting the presence/absence of the analyte(s) and/or for enumerating the analyte(s) in a sample. The high-concentration, low-volume aliquots of the sample that are present in the microstructures can also facilitate enumerating the analyte(s) of interest.

In some embodiments, the analyte of interest can be a microorganism of interest itself, and in some embodiments, the analyte can be an indicator of a viable microorganism of interest. In some embodiments, the present disclosure can include systems and methods for determining the presence/absence of microorganism(s) of interest in a sample by interrogating the sample for analyte(s) of interest that are representative of the microorganism(s).

In some embodiments, rapid detection can refer to detection in no greater than 24 hours, no greater than 20 hours, no greater than 16 hours, no greater than 12 hours, no greater than 8 hours, no greater than 6 hours, no greater than 5 hours, no greater than 4 hours, or no greater than 3 hours. The detection time, however, can be dependent upon the type of analyte being detected because some microorganisms grow more quickly than others and will therefore reach a detectable threshold more rapidly. One of skill in the art will understand how to identify the appropriate assays (e.g., including the appropriate enzymes and enzymes substrates) to detect an analyte (e.g., microorganism) of interest. However, no matter which assay is used, or which analyte is selected, for a given analyte of interest, the systems and methods of the present disclosure will generally achieve a time-to-result more quickly than that achieved with standard culture techniques (e.g., growth-based detection in a microtiter plate (e.g., 96-well). That is, the systems and methods of the present disclosure can detect the anlayte at least 50% faster than standard culture techniques, for example, where each well contains 100 microliters of a sample), in some embodiments, at least 75% faster, and in some embodiments, at least 90% faster.

Such samples to be analyzed for an analyte of interest can be obtained in a variety of ways. For example, in some embodiments, the sample to be analyzed itself is a liquid sample, such as a dilute liquid sample and/or a dilute aqueous sample. In some embodiments, the sample can include the liquid resulting from washing or rinsing a source of interest (e.g., a surface, fomite, etc.) with a diluent. In some embodiments, the sample can include the filtrate resulting from filtering or settling a liquid composition resulting from combining a source of interest with an appropriate diluent. That is, large insoluble matter and/or matter having a lower or higher density than the analyte(s) of interest, such as various foods, fomites, or the like, can be removed from a liquid composition in a first filtration or settling step to form the sample that will be analyzed using a method of the present disclosure.

The term “source” can be used to refer to a food or nonfood desired to be tested for analytes. The source can be a solid, a liquid, a semi-solid, a gelatinous material, and combinations thereof. In some embodiments, the source can be provided by a substrate (e.g., a swab or a wipe) that was used, for example, to collect the source from a surface of interest. In some embodiments, the liquid composition can include the substrate, which can be further broken apart (e.g., during an agitation or dissolution process) to enhance retrieval of the source and any analyte of interest. The surface of interest can include at least a portion of a variety of surfaces, including, but not limited to, walls (including doors), floors, ceilings, drains, refrigeration systems, ducts (e.g., airducts), vents, toilet seats, handles, doorknobs, handrails, bedrails (e.g., in a hospital), countertops, tabletops, eating surfaces (e.g., trays, dishes, etc.), working surfaces, equipment surfaces, clothing, etc., and combinations thereof. All or a portion of the source can be used to obtain a sample that is to be analyzed using the methods of the present disclosure. For example, a “source” can be a water supply or water moving through a pipeline, and a relatively large volume sample can be taken from that source to form a sample that will be tested with the systems and methods of the present disclosure. Therefore, the “sample” can also be from any of the above-described sources.

The term “food” is generally used to refer to a solid, liquid (e.g., including, but not limited to, solutions, dispersions, emulsions, suspensions, etc., and combinations thereof) and/or semi-solid comestible composition. Examples of foods include, but are not limited to, meats, poultry, eggs, fish, seafood, vegetables, fruits, prepared foods (e.g., soups, sauces, pastes), grain products (e.g., flour, cereals, breads), canned foods, milk, other dairy products (e.g., cheese, yogurt, sour cream), fats, oils, desserts, condiments, spices, pastas, beverages, water, animal feed, drinking water, other suitable comestible materials, and combinations thereof.

The term “nonfood” is generally used to refer to sources of interest that do not fall within the definition of “food” and are generally not considered to be comestible. Examples of nonfood sources can include, but are not limited to, clinical samples, cell lysates, whole blood or a portion thereof (e.g., serum), other bodily fluids or secretions (e.g., saliva, sweat, sebum, urine), feces, cells, tissues, organs, biopsies, plant materials, wood, soil, sediment, medicines, cosmetics, dietary supplements (e.g., ginseng capsules), pharmaceuticals, fomites, other suitable non-comestible materials, and combinations thereof.

The term “fomite” is generally used to refer to an inanimate object or substrate capable of carrying infectious organisms and/or transferring them. Fomites can include, but are not limited to, cloths, mop heads, towels, sponges, wipes, eating utensils, coins, paper money, cell phones, clothing (including shoes), doorknobs, feminine products, diapers, etc., portions thereof, and combinations thereof.

The term “analyte” is generally used to refer to a substance to be detected (e.g., by a laboratory or field test). A sample can be tested for the presence, quantity and/or viability of particular analytes. Such analytes can be present within a source (e.g., on the interior), or on the exterior (e.g., on the outer surface) of a source. Examples of analytes can include, but are not limited to, microorganisms, biomolecules, chemicals (e.g. pesticides, antibiotics), metal ions (e.g. mercury ions, heavy metal ions), metal-ion-containing complexes (e.g., complexes comprising metal ions and organic ligands), enzymes, coenzymes, enzyme substrates, indicator dyes, stains, adenosine triphophate (ATP), adenosine diphophate (ADP), adenylate kinase, luciferase, luciferin, and combinations thereof.

A variety of testing methods can be used to identify or quantitate an analyte of interest, including, but not limited to, microbiological assays, biochemical assays (e.g. immunoassay), or a combination thereof. In some embodiments, analytes of interest can be detected genetically; immunologically; colorimetrically; fluorimetrically; luminetrically; by detecting an enzyme released from a live cell in the sample; by detecting light that is indicative of the analyte of interest; by detecting light by absorbance, reflectance, fluorescence, or combinations thereof; or combinations thereof. That is, in some embodiments, interrogating the sample (or a concentrate of the sample) includes optically interrogating the sample, which can include any of the above-described types of optical interrogation, or any described below.

Specific examples of testing methods that can be used include, but are not limited to, antigen-antibody interactions, molecular sensors (affinity binding), thermal analysis, microscopy (e.g., light microscopy, fluorescent microscopy, immunofluorescent microscopy, scanning electron microscopy (SEM), transmission electron microscopy (TEM)), spectroscopy (e.g., mass spectroscopy, nuclear magnetic resonance (NMR) spectroscopy, Raman spectroscopy, infrared (IR) spectroscopy, x-ray spectroscopy, attenuated total reflectance spectroscopy, Fourier transform spectroscopy, gamma-ray spectroscopy, etc.), spectrophotometry (e.g., absorbance, reflectance, fluorescence, luminescence, colorimtetric detection etc.), electrochemical analysis, genetic techniques (e.g., polymerase chain reaction (PCR), transcription mediated amplification (TMA), hybridization protection assay (HPA), DNA or RNA molecular recognition assays, etc.), adenosine triphosphate (ATP) detection assays, immunological assays (e.g., enzyme-linked immunosorbent assay (ELISA)), cytotoxicity assays, viral plaque assays, techniques for evaluating cytopathic effect, other suitable analyte testing methods, or a combination thereof.

The term “microorganism” is generally used to refer to any archaea, prokaryotic or eukaryotic microscopic organism, including without limitation, one or more of bacteria (e.g., motile or vegetative, Gram positive or Gram negative), viruses (e.g., Norovirus, Norwalk virus, Rotavirus, Adenovirus, DNA viruses, RNA viruses, enveloped, non-enveloped, human immunodeficiency virus (HIV), human Papillomavirus (HPV), etc.), bacterial spores or endospores, algae, fungi (e.g., yeast, filamentous fungi, fungal spores), prions, mycoplasmas, and protozoa. Examples of bacteria can include, but are not limited to, SRB. SRBs include psychrophiles, mesophiles and thermophiles. They have been isolated from a variety of ecosystems. Particular examples of SRB can include, but are not limited to, Archaeoglobus spp., Archaeoglobus fulgidus, A. profundus, Balnearium lithotrophicum, Desulfacinum spp., Desulfarculus spp., Desulfarculus baarsii, Desulfobacca spp., Desulfobacter spp., Desulfobacter curvatus, Desulfobacter giganteus, Desulfobacter halotolerans, Desulfobacter hydrogenophilus, Desulfobacter latus, Desulfobacter postgatei, Desulfobacter vibrioformis, Desulfocapsa spp., Desulforhopalus spp., Desulforhopalus singaporensis, Desulforhopalus vacuolatus, Desulfobacterium spp., Desulfobacterium anilini, Desulfitrobacterium atlanticum, Desulfobacterium autotrophicum, Desulfobacterium catecholicum, Desulfobacterium cetonicum, Desulfobacterium indolicum, Desulfobacterium macestii, Desulfobacterium niacin, Desulfurobacterium pacificum, Desulfobacterium phenolicum, Desulfurobacterium thermolithotrophum, Desulfobacterium vacuolatum, Desulfobacula spp., Desulfobotulus spp., Desulfobotulus sapovorans, Desulfocella spp., Desulfobulbus spp., Desulfobulbus alkaphilus, Desulfobulbus elongates, Desulfobulbus japonicas, Desulfobulbus marinus, Desulfobulbus mediterraneus, Desulfobulbus propionicus, Desulfobulbus rhabdoformis, Desulfocapsa, Desulfococcus, Desulfocurvus vexinensis, Desulfofaba spp., Desulfofaba gelida, Desulfofaba fastidiosa, Desulfofaba hansenii, Desulfofrigus spp., Desulfofrigus oceanense, Desulfofrigus fragile, Desulfofustis spp., Desulfohalobium spp., Desulfohalobium retbaense, Desulfomicrobium spp., Desulfomicrobium baculatum, Desulfomicrobium apsheronum, Desulfomicrobium escambiense, Desulfomicrobium thermophilum, Desulfomicrobium orate, Desulfomicrobium norvegicum, Desulfomicrobium macestii, Desulfomonile spp., Desulfomonile limimaris, Desulfomonile tiedjei, Desulfomusa spp., Desulforhopalus spp., Desulfotalea spp., Desulfotalea arctica, Desulfotalea psychrophila, Desulfomonas spp., Desulfuromusa spp., Desulfuromusa bakii, Desulfuromusa ferrireducens, Desulfuromusa kysingii, Desulfuromusa succinoxidans, Desulfonema spp., Desulfonema ishimotonii, Desulfonema limicola, Desulfonema magnum, Desulfonatronobacter spp., Desulfonatronobacter acetoxydans, Desulfonatronobacter acidivorans, Desulfonatronum spp., Desulfonatronum alkalitolerans, Desulfonatronum buryatense, Desulfonatronum cooperativum, Desulfonatronum lacustre, Desulfonatronum thioautotrophicum, Desulfonatronum thiodismutans, Desulfonatronum thiosulfatophilum, Desulfonatronovibrio spp., Desulfonatronovibrio halophilus, Desulfonatronovibrio hydrogenovorans, Desulfonatronovibrio magnus, Desulfonatronovibrio thiodismutans, Desulfopila aestuarii, Desulfosarcina, Desulfosporosinus spp., Desulfosporosinus acididurans, Desulfosporosinus acidophilus, Desulfosporosinus auripigmenti, Desulfosporosinus burensis, Desulfosporosinus hippie, Desulfosporosinus lacus, Desulfosporosinus meridiei, Desulfosporosinus orientis, Desulfosporosinus youngiae, Desulfotalea spp, Desulfotalea psychrophila, Desulfotignum, Desulfotignum balticum, Desulfotignum phosphitoxidans, Desulfotignum toluenicum, Desulfotomaculum spp., Desulfotomaculum acetoxidans, Desulfotomaculum aeronauticum, Desulfotomaculum alcoholivorax, Desulfotomaculum Desulfotomaculum antarcticum, Desulfotomaculum arcticum, Desulfotomaculum auripigmentum, Desulfotomaculum australicum, Desulfotomaculum carboxydivorans, Desulfotomaculum defluvii, Desulfotomaculum geothermicum, Desulfotomaculum gibsoniae, Desulfotomaculum guttoideum, Desulfotomaculum halophilum, Desulfotomaculum hydrothermale, Desulfotomaculum intricatum, Desulfotomaculum kuznetsovii, Desulfotomaculum luciae, Desulfotomaculum nigrificans, Desulfotomaculum orientis, Desulfotomaculum peckii, Desulfotomaculum putei, Desulfotomaculum ruminis, Desulfotomaculum sapomandens, Desulfotomaculum solfataricum, Desulfotomaculum the rmoacetoxidans, Desulfotomaculum thermobenzoicum, Desulfotomaculum thermobenzoicum subsp. Thermobenzoicum, Desulfotomaculum thermobenzoicum subsp. Thermosyntrophicum, Desulfotomaculum thermosapovorans, Desulfotomaculum the rmosubterraneum, Desulfotomaculum tongense, Desulfotomaculum varum, Desulforhabdus, Desulfospira, Desulfuromonas, Desulfovibrio spp., Desulfovibrio acrylicus, Desulfovibrio aerotolerans, Desulfovibrio aespoeensis, Desulfovibrio africanus, Desulfovibrio africanus subsp. africanus, Desulfovibrio africanus subsp. Uniflagellum, Desulfovibrio alaskensis, Desulfovibrio alcoholivorans, Desulfovibrio alkalitolerans, Desulfovibrio aminophilus, Desulfovibrio arcticus, Desulfovibrio baarsii, Desulfovibrio baculatus, Desulfovibrio bastinii, Desulfovibrio biadhensis, Desulfovibrio bizertensis, Desulfovibrio burkinensis, Desulfovibrio butyratiphilus, Desulfovibrio capillatus, Desulfovibrio carbinolicus, Desulfovibrio carbinoliphilus, Desulfovibrio cavernae, Desulfovibrio cuneatus, Desulfovibrio dechloracetivorans, Desulfovibrio desulfuricans, Desulfovibrio desulfuricans subsp. aestuarii, Desulfovibrio desulfuricans subsp. desulfuricans, Desulfovibrio ferrireducens, Desulfovibrio (rigidus, Desulfovibrio fructosivorans, Desulfovibrio furfuralis, Desulfovibrio gabonensis, Desulfovibrio giganteus, Desulfovibrio gigias, Desulfovibrio gracilis, Desulfovibrio halophilus, Desulfovibrio hydrothermalis, Desulfovibrio idahonensis, Desulfovibrio indonesiensis, Desulfovibrio inopinatus, Desulfovibrio intestinalis, Desulfovibrio legallii, Desulfovibrio litoralis, Desulfovibrio longreachensis, Desulfovibrio longus, Desulfovibrio magneticus, Desulfovibrio marinus, Desulfovibrio marinisediminis, D esulfovibrio marrakechensis, Desulfovibrio mexicanus, Desulfovibrio oceani, Desulfovibrio oceani subsp. galateae, Desulfovibrio paquesii, Desulfovibrio piezophilus, Desulfovibrio pi ger, Desulfovibrio portus, Desulfovibrio profundus, Desulfovibrio psychrotolerans, Desulfovibrio putealis, Desulfovibrio salexigens, Desulfovibrio sapovorans, Desulfovibrio senezii, Desulfovibrio simplex, Desul fovibrio sulfodismutans, Desulfovibrio termitidis, Desulfovibrio thermophiles, Desulfovibrio tunisiensis, Desulfovibrio vietnamensis, Desulfovibrio vulgaris, Desulfovibrio vulgaris subsp. oxamicus, Desulfovibrio vulgaris subsp. vulgaris, Desulfovibrio zosterae, Desulfurella spp., Desulfurella acetivorans, Desulfurella kamchatkensis, Desulfurella multipotens, Desulfurella propionica, Dethiosulfovibrio spp., Dethiosulfovibrio acidaminovorans, Dethiosulfovibrio marinus, Dethiosulfovibrio peptidovorans, Dethiosulfovibrio russensis, Dethiosulfovibrio salsuginis, Thermodesulfobacterium spp., Thermodesulfobacterium commune, Thermodesulfobacterium hveragerdense, Thermodesulfobacterium hydrogeniphilum, Thermodesulfobacterium mobile, Thermodesulfobacterium thermophilum, Thermodesulfovibrio spp., Thermodesulfovibrio aggregans, Thermodesulfovibrio hydrogeniphilus, Thermodesulfovibrio islandicus, Thermodesulfovibrio thiophilus, Thermodesulfovibrio yellowstonii, Thermodesulforhabdus, Thermovibrio guaymasensis, Syntrophobacter spp., Syntrophobacter fumaroxidans, Syntrophobacter pfennigii, Syntrophobacter sulfatireducens, Syntrophobacter wolinii.

The term “biomolecule” is generally used to refer to a molecule, or a derivative thereof, that occurs in or is formed by an organism. For example, a biomolecule can include, but is not limited to, at least one of an amino acid, a nucleic acid, a polypeptide, a protein, a polynucleotide, a lipid, a phospholipid, a saccharide, a polysaccharide, and combinations thereof. Specific examples of biomolecules can include, but are not limited to, a metabolite (e.g., staphylococcal enterotoxin), an allergen (e.g., peanut allergen(s), egg allergen(s), pollens, dust mites, molds, danders, or proteins inherent therein, etc.), a hormone, a toxin (e.g., Bacillus diarrheal toxin, aflatoxin, Clostridium difficile toxin etc.), RNA (e.g., mRNA, total RNA, tRNA, etc.), DNA (e.g., plasmid DNA, plant DNA, etc.), a tagged protein, an antibody, an antigen, ATP, and combinations thereof.

The terms “soluble matter” and “insoluble matter” are generally used to refer to matter that is relatively soluble or insoluble in a given medium, under certain conditions. Specifically, under a given set of conditions, “soluble matter” is matter that goes into solution and can be dissolved in the solvent (e.g., diluent) of a system. “Insoluble matter” is matter that, under a given set of conditions, does not go into solution and is not dissolved in the solvent of a system. A source, or a sample taken from that source, can include soluble matter and insoluble matter (e.g., cell debris). Insoluble matter is sometimes referred to as particulate(s), precipitate(s), or debris and can include portions of the source material itself (i.e., from internal portions or external portions (e.g., the outer surface) of the source) or other source residue or debris resulting from an agitation process. In addition, a liquid composition comprising the source and a diluent can include more dense matter (i.e., matter having a higher density than the diluent and other matter in the mixture) and less dense matter (i.e., matter having a lower density than the diluent and other matter in the mixture). As a result, a diluent of the sample can be selected, such that the analyte(s) of interest is(are) more dense than the diluent and can be concentrated via settling (e.g., centrifugation). The term “diluent” is generally used to refer to a liquid added to a source material to disperse, dissolve, suspend, emulsify, wash and/or rinse the source. A diluent can be used in forming a liquid composition, from which a sample to be analyzed using the methods of the present disclosure can be obtained. In some embodiments, the diluent is a sterile liquid. In some embodiments, the diluent can include a variety of additives, including, but not limited to, surfactants, or other suitable additives that aid in dispersing, dissolving, suspending or emulsifying the source for subsequent analyte testing; rheological agents; antimicrobial neutralizers (e.g., that neutralize preservatives or other antimicrobial agents); enrichment or growth medium comprising nutrients (e.g., that promote selective growth of desired microorganism(s)) and/or growth inhibitors (e.g., that inhibit the growth of undesired microorganism(s)); pH buffering agents; enzymes; indicator molecules (e.g. pH or oxidation/reduction indicators); spore germinants; an agent to neutralize sanitizers (e.g., sodium thiosulfate neutralization of chlorine); an agent intended to promote bacterial resuscitation (e.g., sodium pyruvate); stabilizing agents (e.g., that stabilize the analyte(s) of interest, including solutes, such as sodium chloride, sucrose, etc.); or a combination thereof. In some embodiments, the diluent can include sterile water (e.g., sterile double-distilled water (ddH₂O)); one or more organic solvents to selectively dissolve, disperse, suspend, or emulsify the source; aqueous organic solvents, or a combination thereof. In some embodiments, the diluent is a sterile buffered solution (e.g., Butterfield's Buffer, available from Edge Biological, Memphis Tenn.). In some embodiments, the diluent is a selective or semi-selective nutrient formulation, such that the diluent may be used in the selective or semi-selective growth of the desired analyte(s) (e.g., bacteria). In such embodiments, the diluent can be incubated with a source for a period of time (e.g., at a specific temperature) to promote such growth and/or development of the desired analyte(s).

Examples of growth medium can include, but are not limited to, Tryptic Soy Broth (TSB), Buffered Peptone Water (BPW), Universal Pre-enrichment Broth (UPB), Listeria Enrichment Broth (LEB), Lactose Broth, Bolton broth, or other general, non-selective, or mildly selective media known to those of ordinary skill in the art. The growth medium can include nutrients that support the growth of more than one desired microorganism (i.e., analyte of interest).

Examples of growth inhibitors can include, but are not limited to, bile salts, sodium deoxycholate, sodium selenite, sodium thiosulfate, sodium nitrate, lithium chloride, potassium tellurite, sodium tetrathionate, sodium sulphacetamide, mandelic acid, selenite cysteine tetrathionate, sulphamethazine, brilliant green, malachite green oxalate, crystal violet, Tergitol 4, sulphadiazine, amikacin, aztreonam, naladixic acid, acriflavine, polymyxin B, novobiocin, alafosfalin, organic and mineral acids, bacteriophages, dichloran rose bengal, chloramphenicol, chlortetracycline, certain concentrations of sodium chloride, sucrose and other solutes, and combinations thereof.

The term “agitate” and derivatives thereof is generally used to describe the process of giving motion to a liquid composition, for example, to mix or blend the contents of such liquid composition. A variety of agitation methods can be used, including, but not limited to, manual shaking, mechanical shaking, ultrasonic vibration, vortex stirring, manual stirring, mechanical stirring (e.g., by a mechanical propeller, a magnetic stirbar, or another agitating aid, such as ball bearings), manual beating, mechanical beating, blending, kneading, and combinations thereof.

The term “filtering” is generally used to refer to the process of separating matter by size, charge and/or function. For example, filtering can include separating soluble matter and a solvent (e.g., diluent) from insoluble matter, or filtering can include separating soluble matter, a solvent and relatively small insoluble matter from relatively large insoluble matter. As a result, a liquid composition can be “pre-filtered” to obtain a sample that is to be analyzed using the methods of the present disclosure. A variety of filtration methods can be used, including, but not limited to, passing the liquid composition (e.g., comprising a source of interest, from which a sample to concentrated can be obtained) through a filter, other suitable filtration methods, and combinations thereof.

“Settling” is generally used to refer to the process of separating matter by density, for example, by allowing the more dense matter in the liquid composition (i.e., the matter having a higher density than the diluent and other matter in the mixture) to settle or sink and/or by allowing the less dense matter in the liquid composition (i.e., the matter having a lower density than the diluent and other matter in the mixture) to rise or float. Settling may occur by gravity or by centrifugation. The more dense matter can then be separated from the less dense matter (and diluent) by aspirating the less dense (i.e., unsettled or floating) and diluent from the more dense matter, decanting the less dense matter and diluent, or a combination thereof. Pre-settling steps can be used in addition to or in lieu of pre-filtering steps to obtain a sample that is to be concentrated using the sample detection systems and methods of the present disclosure.

A “filter” is generally used to describe a device used to separate the soluble matter (or soluble matter and relatively small insoluble matter) and solvent from the insoluble matter (or relatively large insoluble matter) in a liquid composition and/or to filter a sample during sample concentration. Examples of filters can include, but are not limited to, a woven or non-woven mesh (e.g., a wire mesh, a cloth mesh, a plastic mesh, etc.), a woven or non-woven polymeric web (e.g., comprising polymeric fibers laid down in a uniform or nonuniform process, which can be calendered), a surface filter, a depth filter, a membrane (e.g., a ceramic membrane (e.g., ceramic aluminum oxide membrane filters available under the trade designation ANOPORE from GE Healthcare Bio-Sciences, Pittsburgh, Pa.), a polycarbonate membrane (e.g., track-etched polycarbonate membrane filters available under the trade designation NUCLEOPORE from GE Healthcare Bio-Sciences)), a polyester membrane (e.g., comprising track-etched polyester, etc.), a sieve, glass wool, a frit, filter paper, foam, etc., and combinations thereof.

In some embodiments, the filter can be configured to separate a microorganism of interest from a sample, for example, by size, charge, and/or affinity. For example, in some embodiments, the filter can be configured to retain a microorganism of interest, such that a filtrand retained on the filter comprises the microorganism of interest.

Additional examples of suitable filters are described in co-pending PCT Publication No. WO2011/156251 (Rajagopal, et al.), which claims priority to U.S. Patent Application No. 61/352,229; PCT Publication No. WO2011/156258 (Mach et al.), which claims priority to U.S. Patent Application No. 61/352,205; PCT Publication No. WO2011/152967 (Zhou), which claims priority to US Patent Application Nos. 61/350,147 and 61/351,441; and PCT Publication No. WO2011/153085 (Zhou), which claims priority to US Patent Application Nos. 61/350,154 and 61/351,447, all of which are incorporated herein by reference in their entirety.

In some embodiments, the term “filtrate” is generally used to describe the liquid remaining after the insoluble matter (or at least the relatively large insoluble matter) has been separated or removed from a liquid composition. In some embodiments, the term “supernatant” is generally used to describe the liquid remaining after the more dense matter has been separated or removed from a liquid composition. Such a filtrate and/or supernatant can form a sample to be used in the present disclosure. Examples of pre-filtration systems and methods that can be used to form a sample for the present disclosure are described in co-pending U.S. Patent Application No. 61/503,356, filed on Jun. 30, 2011, which is incorporated herein by reference in its entirety. In some embodiments, the filtrate and/or supernatant can be incubated for a period of time to grow a microorganism of interest, and the resulting incubated filtrate and/or supernatant can form a sample to be used in the present disclosure. In some embodiments, growth media can be added to aid in growing the microorganism of interest.

In some embodiments, the term “filtrand” is generally used to describe the solid remaining after a liquid source (e.g., water to be tested) has been filtered to separate insoluble matter from soluble matter. Such a filtrand can be further diluted, and optionally agitated, grown (e.g., by adding growth media), and/or incubated, to form a sample to be used in the present disclosure. The filtrand may be present on one surface or side of the filter, and/or may have penetrated at least partially into the depth of the filter. As a result, in some embodiments, a diluent comprising an elution solution, a wash solution, or the like can be used to facilitate removing the filtrand from the filter. In some embodiments, surface filters can be preferred (e.g., over depth filters) for facilitating and enhancing removal of the filtrand from the filter.

In some cases, the retained analyte(s) of interest (e.g., microorganisms) can be eluted from the filter by repositioning the filter so that the force of gravity causes the retained biological organisms to dislodge and thereby elute from the filter. In other cases, retained analyte(s) may be eluted from the filter by manually shaking the filter to dislodge the retained analyte(s) from the filter. In other cases, retained analyte(s) may be eluted by vortexing the filter to dislodge the retained analyte(s) from the filter. In other cases, analyte(s) may be eluted from the filter by foam elution.

In some embodiments, no matter what form the starting sample is in, or how it was obtained, the sample can be agitated, grown (e.g., by adding growth media), and/or incubated, to form a sample to be analyzed by systems and methods of the present disclosure. In some embodiments, various reagents can be added at various stages of the process, including, but not limited to being added to the original sample, being added to the filtrand (e.g., with a diluent) or supernatant used to form the sample to be tested, being coated and/or dried in microstructured recesses that will serve as the detection vessels for a concentrate of the sample, or combinations thereof.

In some embodiments, the term “sediment” is generally used to describe the “pellet” or solid that is separated from the supernatant after the more dense matter has been separated or removed from a liquid composition, for example via centrifugation.

The term “microstructure” or “microstructured feature,” and derivatives thereof, is generally used to refer to a structure or a feature having a structure that is a recognizable geometric shape that either protrudes (e.g., a wall) or is depressed (e.g., a well defined at least partially by the wall). For example, a microstructure can include a microstructured well formed to retain a liquid, a solid, a semi-solid, a gelatinous material, another suitable material, or a combination thereof. A microstructure can also include a wall or a base that at least partially defines a microstructured well. Furthermore, a microstructure can include a protrusion, a recess, or the like that is present on any of the above-described microstructures. For example, a microstructured well or wall can be textured, and such textures can also be referred to as microstructures.

In some embodiments, “microstructured” can refer to features that are no greater than 1000 micrometers in at least two of the possible dimensions, in some embodiments, no greater than 500 micrometers, and in some embodiments, no greater than 200 micrometers. However, in some embodiments of the present disclosure, “microstructured features” can be any features that are sufficient to retain a portion of a sample (e.g., a liquid concentrate of a sample after centrifugation toward a microstructured surface comprising the microstructured features) under normal gravitational forces, at any orientation. Therefore, the microstructured features of the present disclosure can have a sufficient depth (e.g., z dimension), or ratio (i.e., “aspect ratio”) of a z dimension to an x-y dimension (or vice versa), that provides sufficient force to retain a sample (e.g., a concentrated liquid comprising a sediment of a sample) of a given surface tension. The surface energy of the microstructured feature can be controlled (e.g., modified with a surface treatment) to enhance retention, however, generally, microstructured features of the present disclosure, such as wells, recesses or depressions, can have an aspect ratio that provides the necessary capillary forces to retain a sample of interest.

In some embodiments, the aspect ratio can be at least about 0.1, in some embodiments, at least about 0.25, in some embodiments, at least about 0.5, in some embodiments, at least about 1, in some embodiments, at least about 2, in some embodiments, at least about 5, and in some embodiments, at least about 10. Because, in some embodiments, the x-y dimension of a microstructured feature (e.g., a recess) can change along its depth or z dimension (e.g., if the feature includes a draft angle), the aspect ratio can be the ratio of a z dimension to a “representative” x-y dimension. The representative x-y dimension can be a top dimension (i.e., the x-y dimension at the opening of a recess), a bottom dimension (e.g., the x-y dimension at the base of a recess), a middle dimension (e.g., the x-y dimension at the half-depth position), an average x-y dimension (e.g., averaged along the depth), another suitable representative dimension, or the like.

The term “microstructured surface” is generally used to refer to a surface that comprises microstructures or microstructured features.

The term “microreplicate” and derivatives thereof, is generally used to refer to the production of a microstructured surface through a process where positive structured surface features are formed in a tool (e.g., as posts, pins, protrusion, or the like) that is used to form negative features (e.g., recesses, wells, depressions, or the like) in a material.

The phase “substantially transparent” is generally used to refer to a body or substrate that transmits at least 50% of electromagnetic radiation having wavelengths at a selected wavelength or within a selected range of wavelengths in the ultraviolet to infrared spectrum (e.g., from about 200 nm to about 1400 nm; “UV-IR”), in some embodiments, at least about 75% of a selected wavelength (or range) in the UV-IR spectrum, and in some embodiments, at least about 90% of a selected wavelength (or range) in the UV-IR spectrum.

The phrase “substantially non-transparent” is generally used to refer to a body or substrate that transmits less than 50% of electromagnetic radiation having wavelengths at a selected wavelength or within a selected range of wavelengths in the ultraviolet to infrared spectrum (e.g., from about 200 nm to about 1400 nm; “UV-IR”), in some embodiments, less than 25% of a selected wavelength (or range) in the UV-IR spectrum, and in some embodiments, less than 10% of a selected wavelength (or range) in the UV-IR spectrum.

Various details of “substantially transparent” and “substantially non-transparent” materials are described in PCT Patent Publication No. WO 2011/063332 (Halverson et al.), which is incorporated herein by reference in its entirety.

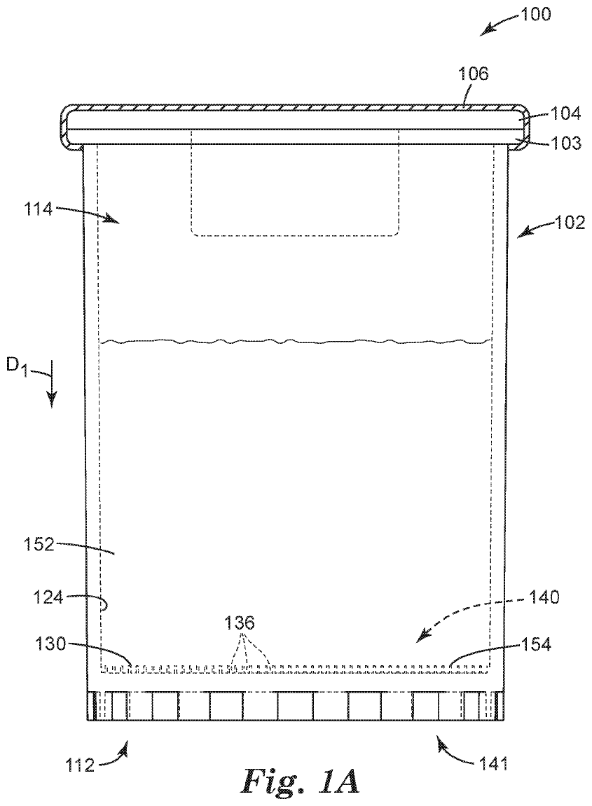

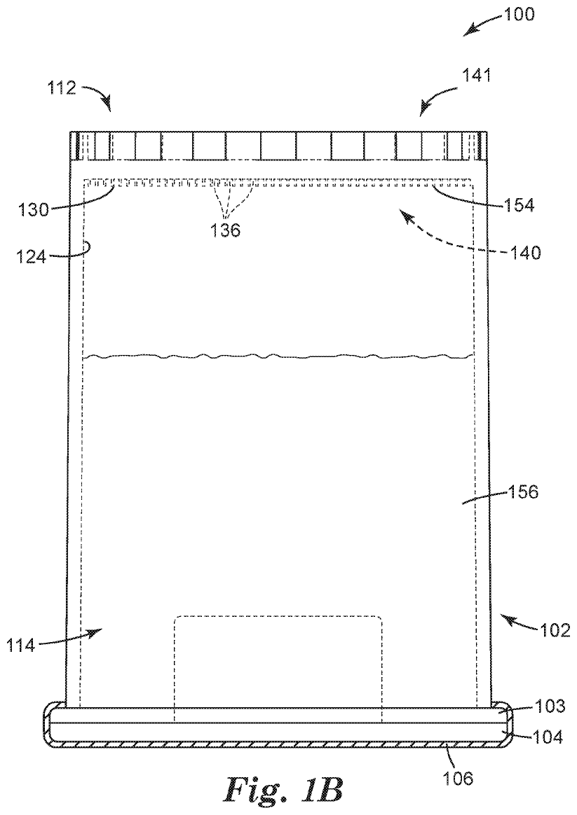

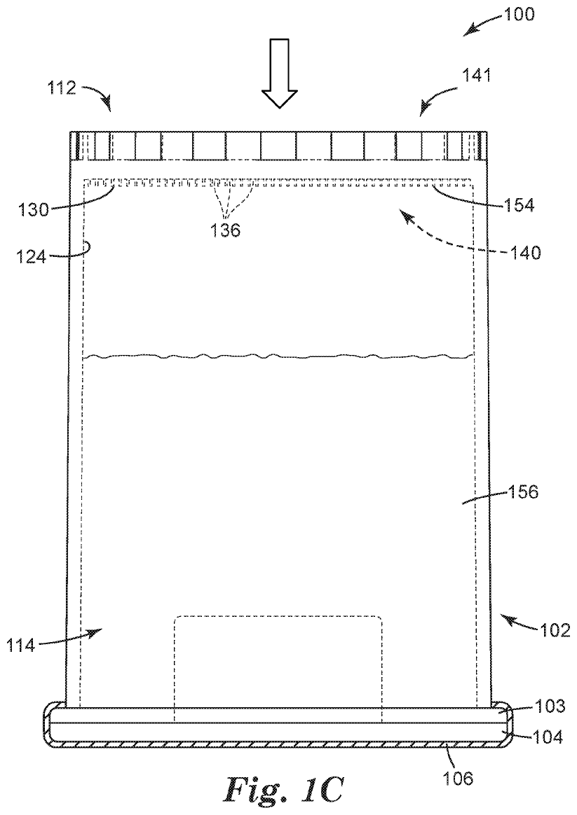

FIGS. 1A-1C illustrates a sample detection system 100 according to one embodiment of the present disclosure. In some embodiments, the sample detection system 100 can be used to interrogate the concentrate for an analyte of interest, that is, for detecting the presence or absence of an analyte of interest.

Various details and features of systems and methods for detecting the presence or absence of an analyte of interest are described in PCT Application Publication No. WO2015/095145 (Rajagopal et al.), which claims priority to U.S. Patent Application No. 61/919,001, both of which are incorporated herein by reference in their entirety. Other systems and methods for detecting the analyte of interest are described in US Patent Application No. 2014/0096598 (Halverson et al.), which are incorporated herein by reference in their entirety.

In some embodiments, the sample detection system 100 can be used to determine the presence or absence of a microorganism of interest in a sample by interrogating the sample for the microorganism itself, or for an analyte of interest that is representative of the presence of the microorganism. For example, in some embodiments, the microorganisms themselves can be concentrated (e.g., sedimented into microstructures by centrifugation) in the sample and then detected in the microstructures, and in some embodiments, analytes that are representative of the presence of microorganisms can be concentrated (e.g., sedimented into microstructures by centrifugation) in the sample and detected in the microstructures. For example, in some embodiments, substrates can be added to the sample (e.g., enzyme substrates) that precipitate after cleavage by the appropriate enzyme. Such precipitated substrates can be concentrated (e.g., sedimented into microstructures by centrifugation, along with the microorganisms/cells) and detected and/or quantified more quickly than they otherwise could be at a low concentration in a large volume sample.

Various examples of analytes are given above, can be detected using fluorescence by concentrating the sample into the microstructures and adding the fluorescent probe, for example, H₂S probe. In the case of precipitated dyes, often the dyes are small molecules that diffuse out of the cells and which may need sufficient incubation time to reach a detectable concentration, even when concentrated in microstructures. The probes can be added either before or after centrifugation, or by having the probes coated and/or dried in the microstructured recesses 136. As a result, a microstructured recess 136 containing a microorganism of interest would be “marked” (e.g., would light up), whereas recesses not containing the microorganism would not be “marked” (e.g., would be dark), and the microorganisms can be detected indirectly.

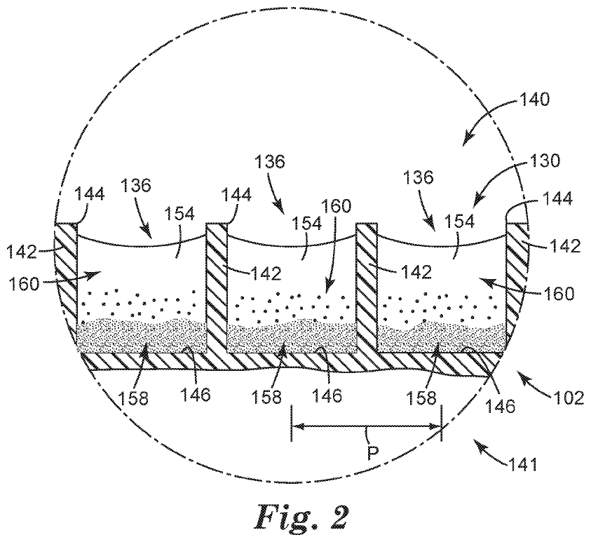

FIGS. 1A-1C and 2 illustrate a sample detection system 100 according to one embodiment of the present disclosure, wherein like numerals represent like elements. The sample detection system 100 of FIGS. 1A-1C and 2 shares many of the same elements, features, and functions.

As shown in FIG. 1A, the sample detection system 100 includes a container 102 adapted to receive a sample 152 that is to be analyzed, for example, for one or more analytes of interest. The sample is generally a liquid sample, in some embodiments, is a dilute liquid sample (i.e., any analyte of interest present in the sample is present at a low concentration), and in some embodiments, is a dilute aqueous sample. The container 102 can be sized and shaped, as desired, to accommodate the sample to be analyzed, and the shape and configuration of the container 102 is shown by way of example only.

The container 102 can be an elongated tube having a closed end or base 112 (e.g., a non-tapered closed end 112) and an open end 114. By way of example only, the container 102 includes a flange or lip 103 which extends from a sidewall close to the open end 114. The flange 103 can facilitate handling, storage and/or transportation of the container 102. A septum or stopper 104 of the system 100 can be coupled to container 102. A snap on stopper is used in the embodiment of FIG. 1A, but it should be understood that any of a variety of mating stopper can be employed to effectively close the container 102. A cap 106 of the system 100 can be placed on the stopper 104 and coupled to container 102. An aluminum seal is crimped on the stopper 104 in the embodiment of FIG. 1A, but it should be understood that any of a variety of mating caps or seals can be employed to effectively seal the container 102. The container 102 can be coupled to any such cap by any of the above-described coupling means, optionally employing one or more seals (e.g., o-rings). In some embodiments, a spacer (not illustrated in FIG. 1A) can be placed between the stopper 104 and cap 106 to provide additional support to the stopper and to compensate for the gap between the stopper and cap.

The open end 114 of the container 102 can be sealed (using for example any of the means described above). In one embodiment, the open end 114 of the container 102 can be sealed with a resealable septum or stopper. A resealable septum or stopper can be pierced, for example, by a hypodermic needle, but will reform a seal upon removal of the needle. As such, the resealable septum or stopper allows a means for the addition of liquid materials (e.g. water sample) into the sealed container by using, for example, a syringe with a hypodermic needle. When a cap is used with the septum or stopper, the cap is configured to allow for access to the resealable septum or stopper. For example, the cap can be an aluminum crimp cap with a tear-out or tear-away section. When the tear-out or tear-away section is removed, the resealable septum or stopper is exposed.

In some embodiments, the closed end 112 of the container 102 can include one or more recesses 136 adapted to retain a concentrate of the sample to be analyzed, each recess 136 opening toward the open end 114 of the container 102. Each recess 136 can include at least one of a well, a depression, a channel, and the like, and combinations thereof. In some embodiments, the one or more recesses 136 can include the channels or interstitial spaces between outwardly-projecting microstructures, such as those described in Ylitalo et al., U.S. Pat. No. 6,386,699. In some embodiments, one or more of the recesses 136 can include a surface modification (e.g., such as a hydrophilic/oleophilic surface treatment or coating) to facilitate retaining a concentrate of interest. The recesses 136 need not all be the same shape or size, and in some embodiments, the closed end 112 of the container 102 includes a variety of recesses 136, ranging from microstructured to larger, and having a variety of shapes and configurations. By way of example only, the container 102 is illustrated as including a flat inner surface 124 in which a microstructured surface 130 is formed, such that the container 102 includes a plurality of microstructured recesses 136.

In some embodiments, at least a portion of the inner surface 124 can include a microstructured surface 130. In embodiments employing the microstructured surface 130, the one or more recesses 136 can be microstructured recesses 136, and the microstructured surface 130 can include a variety of microstructured features.

Particularly, the microstructured recesses 136 are formed in a first side 140 of the container 102 that generally faces the interior (or “inside”) of the container 102, and that generally includes the inner surface 124 of the container 102, or a portion thereof. Particularly, the first side 140 can include the inner surface 124 in which the microstructured recesses 136 can be formed, such that the top opening 144 of each microstructured recess 136 opens toward the first side 140 of the container 102, and toward the interior of the container 102 (see FIG. 2). The container 102 can further include a second side 141 that is generally opposite the first side 140. The second side 141 can face outside of the container 102, for example, away from the container 102. As a result, a concentrate retained in the container 102 (i.e., in the microstructured recesses 136) can be interrogated from the second side 141.

As mentioned above with respect to FIG. 1, the microstructured recesses 136 can be formed in the inner surface 124 of the container 102. However, in some embodiments, the microstructured recesses 136 can alternatively, or additionally, be formed in a substrate (or insert or film) that can be coupled to (e.g., positioned against) at least a portion of the inner surface 124 of the container 102. In embodiments employing a substrate (or film), the thickness of the substrate can be at least about 25 micrometers, in some embodiments, at least about 100 micrometers, and in some embodiments, at least about 400 micrometers. In some embodiments, the thickness of the substrate can be no greater than about 2000 micrometers, in some embodiments, no greater than about 1000 micrometers, and in some embodiments, no greater than about 250 micrometers.

In some embodiments, the substrate can be a film that can be formed of a variety of suitable materials, including but not limited to a polyolefins such as polypropylene, polyethylene, or a blend thereof; olefin copolymers (e.g., copolymers with vinyl acetate); polyesters such as polyethylene terephthalate and polybutylene terephthalate; polyamide (Nylon-6 and Nylon-6,6); polyurethanes; polybutene; polylactic acids; polyvinyl alcohol; polyphenylene sulfide; polysulfone; polycarbonates; polystyrenes; liquid crystalline polymers; polyethylene-co-vinylacetate; polyacrylonitrile; cyclic polyolefins; or a combination thereof. In some embodiments, the film can comprise a compound selected from the group consisting of 1-(3-methyl-n-butylamino)-9, 10-anthracenedione; 1-(3-methyl-2-butylamino)-9, 10-anthracenedione; 1-(2-heptylamino)-9, 10-anthracenedione; 1,1,3,3-tetramethylbutyl-9,10-anthracenedione; 1,10-decamethylene-bis-(-1-amino-9, 10-anthracenedione); 1,1-dimethylethylamino-9,10-anthracenedione; and 1-(n-butoxypropylamino)-9,10-anthracenedione. In some embodiments, the film material can include a cured polymer. Such a cured polymer can be derived from a resin selected from the group consisting of acrylate resins, acrylic resins, acrylic-based resins derived from epoxies, polyesters, polyethers, and urethanes; ethylenically unsaturated compounds; aminoplast derivatives having at least one pendant acrylate group; polyurethanes (polyureas) derived from an isocyanate and a polyol (or polyamine); isocyanate derivatives having at least one pendant acrylate group; epoxy resins other than acrylated epoxies; and mixtures and combinations thereof.

As further shown in FIG. 2, the microstructured recesses 136 can be at least partially defined by a plurality of walls 142, and each microstructured recess 136 can be further defined by a base 146. In some embodiments, the walls 142 can be intersecting walls 142 to define individual cavities, rather than channels having a length.

In some embodiments, the one or more microstructured recesses 136 can define microstructured surface (or a microstructured surface) 130. By way of example only, the microstructured surface 130 is illustrated in FIG. 2 as extending across the entire bottom surface of the container 102; however, in some embodiments, the microstructured surface 130 may only be present in a portion of the base of the container 102.

In such embodiments, the microstructured surface 130 can be formed by a variety of methods, including a variety of microreplication methods, including, but not limited to, casting, coating, molding, and/or compressing techniques, other suitable techniques, or combinations thereof. For example, microstructuring of the microstructured surface 130 can be achieved by at least one of (1) casting a molten thermoplastic using a tool having a microstructured pattern, (2) coating of a fluid onto a tool having a microstructured pattern, solidifying the fluid, and removing the resulting film, and/or (3) passing a thermoplastic film through a nip roll to compress against a tool (e.g., male tooling) having a microstructured pattern (i.e., embossing). The tool can be formed using any of a number of techniques known to those skilled in the art, selected depending in part upon the tool material and features of the desired topography. Other suitable techniques include etching (e.g., chemical etching, mechanical etching, reactive ion etching, etc., and combinations thereof), ablation (e.g., laser ablation, etc.), photolithography, stereolithography, micromachining, knurling (e.g., cutting knurling or acid enhanced knurling), scoring, cutting, etc., or combinations thereof.

Alternative methods of forming the microstructured surface 130 include thermoplastic extrusion, curable fluid coating methods, and embossing thermoplastic layers, which can also be cured. Additional information regarding the substrate or film material and various processes for forming the microstructured surface 130 can be found, for example, in Halverson et al., PCT Publication No. WO 2007/070310 and US Publication No. US 2007/0134784; Hanschen et al., US Publication No. US 2003/0235677; Graham et al., PCT Publication No. WO2004/000569; Ylitalo et al., U.S. Pat. No. 6,386,699; and Johnston et al., US Publication No. US 2002/0128578 and U.S. Pat. Nos. 6,420,622, 6,867,342, and 7,223,364, each of which is incorporated herein by reference.

With microreplication, the microstructured surface 130 can be mass produced without substantial variation from product-to-product and without using relatively complicated processing techniques. In some embodiments, microreplication can produce a microstructured recess surface that retains an individual feature fidelity during and after manufacture, from product-to-product, that varies by no more than about 50 micrometers. In some embodiments, the microstructured surface 130 retains an individual feature fidelity during and after manufacture, from product-to-product, which varies by no more than 25 micrometers. In some embodiments, the microstructured surface 130 comprises a topography (i.e., the surface features of an object, place or region thereof) that has an individual feature fidelity that is maintained with a resolution of between about 50 micrometers and 0.05 micrometers, and in some embodiments, between about 25 micrometers and 1 micrometer.

The microstructured recesses 136 are adapted to retain the concentrate 154 resulting from the centrifugation. Each microstructured recess 136 is shown in FIG. 2 as having a generally rectangular cross-sectional shape and as being formed by at least two walls 142 and a base or closed end 146, and each microstructured recess 136 is separated from an adjacent microstructured recess 136 by a wall 142. Each microstructured recess 136 also includes an open end or top opening 144. It should be understood that the microstructured recesses 136 can include a variety of shapes so as to be able to retain the concentrate 154. Said another way, each microstructured recess 136 can be shaped and dimensioned to provide a reservoir, or well, for the concentrate 154. Examples of suitable recess shapes can include, but are not limited to, a variety of polyhedral shapes, parallelepipeds, prismatoids, prismoids, etc., and combinations thereof. For example, the microstructured recesses 136 can be polyhedral, conical, frusto-conical, pyramidal, frusto-pyramidal, spherical, partially spherical, hemispherical, ellipsoidal, dome-shaped, cylindrical, cube-corner shaped, other suitable shapes, and combinations thereof. Furthermore, the recesses 136 can have a variety of cross-sectional shapes (including a vertical cross-section, a horizontal cross-section, or a combination thereof), including, but not limited to, at least one of parallelograms, parallelograms with rounded corners, rectangles, squares, circles, half-circles, ellipses, half-ellipses, triangles, trapezoids, stars, other polygons (e.g., hexagons), other suitable cross-sectional shapes, and combinations thereof.

Furthermore, the microstructured recesses 136 illustrated in FIG. 2 are shown by way of example only as being regularly arranged (e.g., in a cellular array). However, it should be understood that the microstructured recesses 136 can include a variety of regular arrangements or arrays, random arrangements, or combinations thereof. In some embodiments, the microstructured recesses 136 are arranged randomly on a local or smaller scale, but the random arrangements repeat, or are ordered, on a larger scale. Alternatively, in some embodiments, the microstructured recesses 136 are ordered on a smaller scale, but the ordered regions are randomly arranged on a larger scale.

In addition, in the embodiment illustrated in FIG. 2, the walls 142 are all of the same size and shape. However, it should be understood that a variety of other wall shapes are possible. For example, the walls 142 need not include a substantially rectangular cross-sectional shape, but rather can include any of the above-described cross-sectional shapes.

The walls 142 and the microstructured recesses 146 can be characterized by a variety of sizes, dimensions, distances between walls 142 or microstructured recesses 136, relative sizes, etc. The walls 142 generally have dimensions such as thickness, height, length, width, etc. The microstructured recesses 136 generally have volumes with dimensions such as a radius, diameter, height, width, length, etc. Generally, the walls 142 and/or the microstructured recesses 136 are sized, shaped and spaced to retain the concentrate 154 in the microstructured recesses 136 when container 102 is in any orientation (e.g., by capillary forces).

In some embodiments, the walls 142 can have an average thickness of at least about 1 micrometer, in some embodiments, at least about 5 micrometers, and in some embodiments, at least about 10 micrometers. In some embodiments, the walls 142 can have an average thickness of no greater than about 50 micrometers, in some embodiments, no greater than about 30 micrometers, and in some embodiments, no greater than about 20 micrometers.

In some embodiments, the walls 142 can be shaped and/or sized to minimize the area of the top surface of the walls 142 so that any matter collected on the top surface of the walls 142 can be diverted into an adjacent microstructured recess 136. For example, in some embodiments, the walls 142 can include a taper toward the top surface. In some embodiments, the top surface can include a convex shape. In some embodiments, a combination of a taper and a convex shape can be employed. In some embodiments, the top surface is not radiused, but rather is flat; however, the top surface defining the openings 144 of the microstructured recesses 136 are smooth with little to no sharp edges.

In some embodiments, the configuration of the walls 142 and the microstructured recesses 136 in any given region can be chosen such that the average wall or microstructured recess pitch P (i.e., the center to center distance between adjacent walls 142 or microstructured recesses 136, respectively) is at least about 1 micrometer, in some embodiments, at least about 10 micrometers, and in some embodiments, at least about 50 micrometers. In some embodiments, the average wall or microstructured recess pitch P is no greater than about 1000 micrometers, in some embodiments, no greater than about 800 micrometers, in some embodiments, no greater than about 600 micrometers, in some embodiments, no greater than about 500 micrometers, in some embodiments, no greater than about 200 micrometers, in some embodiments, no greater than about 150 micrometers, and in some embodiments, no greater than about 100 micrometers. In some embodiments, the pitch P can range from 50 micrometers to 850 micrometers.

In general, the higher the packing density of the microstructured recesses 136 (e.g., referred to as average microstructured recess density or average well density), generally, the more concentrate 154 a given area of the first side 140 of the container 102 can contain. Also, in some embodiments, if the microstructured surface 130 includes more land area between microstructured recesses 136, it is possible that the denser portions of the sample (e.g., comprising the analyte of interest) can be centrifuged onto a land area. Therefore, in general, higher microstructured recess densities on the microstructured surface 130 would be preferred to afford a higher likelihood of capture.

In some embodiments, the average microstructured recess density is at least about 20 microstructured recesses/cm², in some embodiments, at least about 30 microstructured recesses/cm², in some embodiments, at least about 70 microstructured recesses/cm², in some embodiments, at least about 100 microstructured recesses/cm², in some embodiments, at least about 150 microstructured recesses/cm², in some embodiments, at least about 200 microstructured recesses/cm², in some embodiments, at least about 500 microstructured recesses/cm², in some embodiments, at least about 800 microstructured recesses/cm², in some embodiments, at least about 900 microstructured recesses/cm², in some embodiments, at least about 1000 microstructured recesses/cm², in some embodiments, at least about 2000 microstructured recesses/cm², and in some embodiments, at least about 3000 microstructured recesses/cm². In some embodiments, the microstructured recess density can be about 825 microstructured recesses/cm².

In some embodiments, the average height of the walls 142 or the average depth of the microstructured recesses 136 (i.e., the distance between the closed end, or base, 146 of each microstructured recess 136 and the open end, or top opening, 144 of the microstructured recess 136) is at least about 5 micrometers, in some embodiments, at least about 20 micrometers, and in some embodiments, at least about 30 micrometers. In some embodiments, the average height of the walls 142 or the average depth of the microstructured recesses 136 can be no greater than about 1000 micrometers, in some embodiments, no greater than about 250 micrometers, in some embodiments, no greater than about 100 micrometers, and in some embodiments, no greater than about 50 micrometers. In the embodiment illustrated in FIG. 2, the wall height is substantially the same as the microstructured recess depth; however, it should be understood that this need not be the case. For example, in some embodiments, the microstructured recesses 136 include a portion that is recessed even below the bottom of the walls 142, such that the microstructured recess depth is greater than the wall height. However, even in such embodiments, the above size ranges can apply.

Another way to characterize the walls 142 and the recesses 136 is to describe them in terms of their aspect ratios. An “aspect ratio” of a recess 136 is the ratio of the depth of a recess 136 to the width of the recess 136. An “aspect ratio” of a wall 142 is the ratio of the height of the wall 142 to the width (or thickness) of the wall 142. The aspect ratios of the recesses 136 and/or the walls 142 can include those described above. In some embodiments, the average wall aspect ratio is at least about 0.01, in some embodiments, at least about 0.05, and in some embodiments, at least about 1. In some embodiments, the average wall aspect ratio is no greater than about 15, in some embodiments, no greater than about 10, and in some embodiments, no greater than about 8.

In some embodiments, the average recess volume of the microstructured recesses 136 is at least about 1 picoliter (pL), in some embodiments, at least about 10 pL, in some embodiments, at least about 100 pL, and in some embodiments, at least about 1000 pL (1 nL). In some embodiments, the average recess volume is no greater than about 1,000,000 pL (1 pL), in some embodiments, no greater than about 100,000 pL, in some embodiments, no greater than about 10,000 pL. In some embodiments, the average recess volume ranges from 10 nL (10,000 pL) to 100 nL (100,000 pL).

Whether or not the recesses 136 or the walls 142 are themselves microstructured, the microstructured surface 130 that includes additional microstructured features, such as protrusions, depressions or recesses, or a combination thereof. At least some of the microstructured features can be formed on a nano-, micro- or macro-scale. Each microstructured feature can be defined by two or more dimensions. The microstructured features can have a desired characteristic size (e.g., length, width, depth, radius, diameter, or other dimension measured along any direction) and density (e.g., features per unit area of the microstructured surface 130). A feature can be configured such that its characteristic length in all three directions (e.g., x, y (in the plane of the microstructured surface 130) and z (into/out of the plane of the microstructured surface 130)) is similar. Alternatively, a feature can be configured such that the characteristic length in one or more directions is greater than in the other directions.

In some embodiments, a feature can have a maximum characteristic length in one or more dimensions of no greater than about 500 micrometers. In some embodiments, the maximum characteristic length is 50 micrometers, and in some embodiments, the maximum characteristic length is 10 micrometers. In some embodiments, the minimum characteristic length in one or more dimensions is 1 nanometer. In some embodiments, the minimum characteristic length is 10 nanometers, and in some embodiments, the minimum characteristic length is 100 nanometers. Furthermore, in some embodiments, the feature density is at least 100 features per square millimeter (mm²), in some embodiments, at least 1,000 features per mm², and in some embodiments, at least 10,000 features per mm².

In general, a sample detection method can be performed using the sample detection system 100 of FIGS. 1A-1C and 2. As shown in FIG. 1A, a septum or stopper 104 can be coupled to container 102 to close the container 102. A cap 106 can be placed, for example, by crimping, on the stopper 104 and coupled to container 102 to seal the container 102. In some embodiments, the container 102 can be flushed with an inert gas, for example, N₂ and pressurized. In some embodiments, the container 102 can be pressurized to at least 1 psi, at least 2 psi, at least 3 psi, at least 5 psi or at least 10 psi. In some embodiments, the container 102 can be pressurized up to 20 psi, up to 15 psi, or up to 10 psi. A sample 152 can be positioned in the container 102. In some embodiments, the container 102 can have a probe, for example, H₂S probe and an enzyme substrate in the container. Alternatively, the probe and enzyme substrate can be added into the container 102 in a separate step.

Any suitable probe for detecting an analyte of interest can be used. For example, H₂S probes, which can react with H₂S to form iron(II) sulfide, allows for detection of actively growing SRB. In some embodiments, H₂S probe can be used to detect SRB. For example, H₂S probe can react with H₂S to form iron(II) as a black precipitate, which indicates the presence of SRB. In some embodiments, a fluorescent probe to detect H₂S is used. For example, H₂S probe can reacts with H₂S to form a fluorescent product. In some embodiments, H₂S probe can act as a colorimetric or fluorescent indicator. Examples of suitable H₂S probe can include, but are not limited to molecules linked to fluorescent molecules such as fluorescein, BODIPY, coumarin, etc. Commercially available reagents, for example, Washington State Probe-1 (WSP-1, 3′-methoxy-3-oxo-3H-spiro[isobenzofuran-1,9′-xanthen]-6′-yl 2-(pyridin-2-yldisulfanyl)benzoate, Cayman Chemicals, Ann Arbor, Mich.) and AzMC (7-azido-4-methylcoumarin, Sigma-Aldrich, St. Louis, Mo.) can be used to detect H₂S.

Any suitable enzyme substrate for detecting an analyte of interest can be used. For example, dye enzyme substrate for enzyme activity such as esterase, phosphatase, protease, peptidase, peroxidase, etc., allows for detection of actively growing cells. The enzyme substrate can act as a fluorescent indicator. In some embodiments, the enzyme substrate can be a substrate for phosphatase or esterase. In some embodiments, the enzyme substrate can react with a phosphatase or esterase to form a fluorescent product. Examples of suitable enzyme substrate can include, but are not limited to, 4-Methylumbelliferyl phosphate (MUP), 6,8-Difluoro-7-hydroxy-4-methylcoumarin (DiFMU), 6,8-difluoro-4-methylumbelliferyl phosphate (DiFMUP), Fluorescein diphosphate (FDP), 7-Amino-4-methylcoumarin based substrates, 7-Amino-4-chloromethylcoumarin based substrates, 4-Methylumbelliferyl acetate (MU-Ac), 3-(2-Benzoxazolyl)umbelliferyl acetate(BzUA), 5(6)-Carboxyfluoresein diacetate (CFDA), 4-Methylumbelliferyl butyrate (MU-Bu), Fluorescein diacetate (FDA), 2′, 7′-Dichlorofluorescein diacetate (DFDA) and Resorufin acetate (RFA).

The sample detection system 100 (i.e., the container 102) can be centrifuged in a first direction (or orientation) D₁ toward the microstructured recess 136. Such a centrifugation process can form a concentrate 154 of the sample and a supernatant 156, and can cause the concentrate 154 comprising the more dense matter of the sample 152 to be moved into the microstructured recess 136. The concentrate 154 can include a sediment 158 of the sample that is formed as a result of the centrifugation process and a liquid 160 of the sample, which can also include soluble matter, and particularly, soluble matter having a lower density than the sediment 158. The concentrate 154 can also include the H₂S probe and the enzyme substrate. The concentrate 154, and particularly, the sediment 158 (if present) can include the analyte(s) of interest (e.g., the microorganism(s) of interest or an analyte representative of the microorganism(s) of interest), if present in the sample. The liquid 160 can include at least a portion of the supernatant 156 of the sample 152.

In the centrifugation step shown in FIG. 1A, the centrifugation g-force, duration and/or number of cycles necessary to form and retain the concentrate 154 in the microstructured recess 136 can vary depending on one or more of the composition of the sample 152, the analyte(s) of interest, and the like. In some embodiments, the amount of g-force required to concentrate the analyte(s) of interest can depend on the size and density of the analyte, the density and viscosity of the diluent, and the volume of sample in the container 102 (i.e. the height of the sample in the container 102 defines the distance the analyte needs to migrate under a specified g-force to reach the microstructured recess 136). The sedimentation velocity (V, in centimeters per second (cm/s)) can be approximated using Equation 1:

V=2ga²(ρ1−ρ2)/9η (1)

where g=acceleration in cm/s² (i.e., g-force in gs*980 cm/s²), ρ1=analyte density in g/cm³, ρ2=density of sample media (e.g., diluent) in g/cm³, η=coefficient of viscosity in poises (g/cm/s), and a=analyte radius in centimeters (assuming a spherical shape). In some centrifuges, the g-force can be determined by the rotational speed (e.g., in revolutions per minute (RPM)) and the distance of the sample from the center of the rotor (i.e. the sample experiences a higher g-force at the same rotational speed if it is placed further away from the rotor). As a result, in order to collect the analyte(s) of interest that may reside in the sample furthest from the microstructured recess 136, the distance between the center of the rotor and the height of the sample positioned closest to the rotor can be calculated to estimate what the g-force would need to be to move the analyte(s) of interest the furthest distance in the sample 152 to maximize collection of the analyte(s) of interest.

The sedimentation velocity can be calculated using the above equation, and then the centrifugation time (i.e., duration) can be calculated by dividing the distance (e.g., the maximum distance) the analyte(s) of interest, if present, would need to travel, by the sedimentation velocity. Alternatively, the desired time and distance can be used to estimate a sedimentation velocity, and the necessary g-force can then be calculated using Equation 1.

In some embodiments, the g-force in the centrifugation step can be at least about 500·g (e.g., 500*9.8 m/s² on earth, at sea level), in some embodiments, at least about 1000·g, and in some embodiments, at least about 5000·g. In some embodiments, the g-force in the centrifugation step can be no greater than about 100,000·g, in some embodiments, no greater than about 50,000·g, and in some embodiments, no greater than about 10,000·g.

In some embodiments, the duration of the centrifugation step can be at least about 1 minute, in some embodiments, at least about 5 minutes, and in some embodiments, at least about 10 minutes. In some embodiments, the duration of the centrifugation step can be no greater than about 120 minutes, in some embodiments, no greater than about 60 minutes, and in some embodiments, no greater than about 20 minutes.

As shown in FIG. 1B, in some embodiments, the container 102 can then be inverted, e.g., prior to detection, such that at least a portion of supernatant 156 resulting from the centrifugation step is removed from being in contact with the microstructured recess 136, while the concentrate 154 remains retained in the microstructured recess 136. FIG. 2 illustrates a schematic cross-sectional view of a portion of the sample detection system of FIG. 1B, with the concentrate 154 retained in the recesses 136 of the container, after the container is inverted and at least a portion of supernatant 156 resulting from the centrifugation step is removed from the microstructured recess 136. The term “inverted” is used herein to refer to a change in orientation and can include orienting at a variety of angles, and is not limited to changing the orientation by 180 degrees. The microstructured recess 136 can be adapted to retain the concentrate 154 under normal gravitational forces (e.g., under standard gravity, i.e., the standard value of Earth's gravitational acceleration at sea level, 9.8 m/s²).

In some embodiments, the inverting step can include inverting the container 102 by at least 20 degrees (e.g., from −10 degrees to +10 degrees, or from 0 degrees to +20 degrees, etc.), in some embodiments, by at least 45 degrees, in some embodiments, by at least 60 degrees, in some embodiments, by at least 90 degrees, and in some embodiments, by 180 degrees. The speed of inverting the sample detection containers of the present disclosure need not be tightly controlled for the purpose of ensuring that the concentrate 154 is substantially contained in the microstructured recess 136 and/or protected from turbulence as the supernatant 156 is drained away.

As shown in FIG. 1C, the concentrate 154 in the microstructured recess 136 can then be interrogated (e.g., optically interrogated) from the outside or exterior of the container 102, i.e., from the second side 141 of the container 102. It should be understood that the microstructured recess 136 can be interrogated from any desired direction. The container 102, or at least a portion thereof, can be colorless in order to enable interrogating (e.g., optically) the concentrate 154 from the second side 141. Also, such embodiments can employ a container 102 and a cap 106 that are permanently coupled together, because the detection, or interrogation, step can be performed from the outside of the sample detection system 100, such that the cap 164 need not be decoupled from the container 102 for the interrogation step.

The interrogation of the concentrate 154 can include any of the above-described detection methods for detecting an analyte of interest in a sample, including optical interrogation methods, such as optical scanning, imaging, or any of the other methods described above. For example, fluorescent detection can include directing electromagnetic energy toward the concentrate 154 in the microstructured recess 136 at a first frequency, and detecting electromagnetic energy emitted from the concentrate 154 in the microstructured recess 136 at a second frequency. In some embodiments, fluorescent detection can further include directing electromagnetic energy toward the concentrate 154 in the microstructured recess 136 at a third frequency, and detecting electromagnetic energy emitted from the concentrate 154 in the microstructured recess 136 at a fourth frequency. In some embodiments, the first frequency can be the energy for excitation associated with the product from the reaction of the H₂S probe with H₂S and the third frequency can be the energy for excitation associated with the product from the reaction of the enzyme substrate with an enzyme. In some embodiments, the second frequency can be the emitted energy associated with the product from the reaction of the H₂S probe with H₂S and the fourth frequency can be the emitted energy associated with the product from the reaction of the enzyme substrate with an enzyme. By way of further example, colorimetric detection can include emitting electromagnetic energy at the concentrate 154 in the microstructured recess 136 at a broad range of frequencies (i.e., broad-spectrum light), and detecting at least one of the transmittance and the absorbance of at least a portion of the concentrate 154 in the microstructured recess 136.

In some embodiments, the microstructured recess 136 can include a base 146 that is formed by at least a portion of the second side (or second major surface) 141 of the container 102, and which is substantially transparent, such that the contents of the microstructured recess 136 can be visible from the second side 141 of the container 102 (i.e., from the outside of the sample detection system 100). In such embodiments, any sidewalls of the microstructured recess 136 can be substantially non-transparent to inhibit cross-talk between wells, and to enhance detection, particularly, optical detection or interrogation.

In some embodiments, at least a portion of the container 102 can include an optical window that is substantially transparent. The optical window can be at least partially coextensive (i.e., overlapping) with the microstructured recess 136, such that the microstructured recess 136 (and its contents) is visible from the outside of the container 102, and particularly from the second side 141 of the container 102.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure.

All references and publications cited herein are expressly incorporated herein by reference in their entirety into this disclosure.

The following embodiments are intended to be illustrative of the present disclosure and not limiting.

### Embodiments

Embodiment 1 is a method of detecting an analyte of interest comprising:

providing a container adapted to receive a sample, the container comprising a microstructured surface;

positioning the sample in the container;

adding an H₂S probe and an enzyme substrate to the container;

centrifuging the container toward the microstructured surface to form a sediment and a supernatant of the sample;

inverting the container, after centrifuging the container, to remove at least a portion of the supernatant of the sample from being in contact with the microstructured surface, such that a concentrate of the sample is retained in the microstructured surface, the concentrate comprising the sediment; and interrogating the concentrate in the microstructured surface for the analyte of interest.

Embodiment 2 is a method of detecting an analyte of interest comprising:

providing a container adapted to receive a sample, the container having an H₂S probe and an enzyme substrate, wherein the container comprises a microstructured surface;

positioning the sample in the container;

centrifuging the container toward the microstructured surface to form a sediment and a supernatant of the sample;

inverting the container, after centrifuging the container, to remove at least a portion of the supernatant from being in contact with the microstructured surface, such that a concentrate of the sample is retained in the microstructured surface, the concentrate comprising the sediment; and

interrogating the concentrate in the microstructured surface for the analyte of interest.

Embodiment 3 is the method of any of embodiments 1-2, further comprises flushing the container with an inert gas before positioning the sample.
Embodiment 4 is the method of any of embodiments 1-3, further comprises pressurizing the container.
Embodiment 5 is the method of any of embodiments 1-4, wherein the microstructured surface forms at least a portion of an inner surface of the container.
Embodiment 6 is the method of any of embodiments 1-5, wherein at least a portion of the container proximate the microstructured surface is substantially transparent to facilitate interrogating the concentrate from an exterior of the container.
Embodiment 7 is the method of any of embodiments 1-6, wherein the microstructured surface comprises a plurality of microstructured recesses, each recess having a base, and wherein each base is substantially transparent.
Embodiment 8 is the method of embodiment 7, wherein at least one of the plurality of microstructured recesses includes a sidewall, and wherein the sidewall is substantially non-transparent.
Embodiment 9 is the method of embodiment 7, wherein each of the plurality of recesses contains a volume of no greater than 1 microliter.
Embodiment 10 is the method of embodiment 7, wherein the microstructured surface includes a recess density of at least about 100 recesses per square centimeter.
Embodiment 11 is the method of any of embodiments 1-10, wherein the container comprises an open end configured to receive a sample and a closed end, wherein the microstructured surface is formed in a first side of the closed end that is positioned to face the open end during centrifugation, wherein the closed end further comprises a second side opposite the first side.
Embodiment 12 is the method of embodiment 11, wherein at least a portion of the closed end proximate the microstructured surface is substantially transparent.
Embodiment 13 is the method of any of embodiments 1-12, wherein the container further comprises a cap to seal the open end.
Embodiment 14 is the method of any of embodiments 1-13, wherein the container further comprises a septum between the cap and the open end.
Embodiment 15 is the method of any of embodiments 1-14, wherein interrogating the concentrate in the microstructured surface includes optically interrogating concentrate in the microstructured surface.
Embodiment 16 is the method of embodiment 15, wherein optically interrogating includes interrogating the concentrate in the microstructured surface for fluorescence.
Embodiment 17 is the method of embodiment 15 or 16, wherein optically interrogating includes

directing electromagnetic energy toward the concentrate in the microstructured surface at a first frequency, and

detecting electromagnetic energy emitted from the concentrate in the microstructured surface at a second frequency.

Embodiment 18 is the method of embodiment 17, wherein optically interrogating includes interrogating the concentrate colorimetrically.
Embodiment 19 is the method of embodiment 15 or 18, wherein optically interrogating includes

emitting electromagnetic energy at the concentrate in the microstructured surface at a broad range of frequencies, and

detecting at least one of the transmittance and the absorbance of at least a portion of the concentrate in the microstructured surface.

Embodiment 20 is the method of any of embodiments 15-19, wherein optically interrogating the concentrate in the microstructured surface includes optically scanning the microstructured surface.
Embodiment 21 is the method of any of embodiments 15-20, wherein optically interrogating the concentrate in the microstructured surface includes imaging the microstructured surface.
Embodiment 22 is the method of any of embodiments 1-21, wherein interrogating the concentrate in the microstructured surface includes detecting light that is indicative of the presence of the analyte of interest.
Embodiment 23 is the method of any of embodiments 1-22, wherein interrogating the concentrate in the microstructured surface includes detecting light by absorbance, reflectance, or fluorescence.
Embodiment 24 is the method of any of embodiments 1-23, wherein interrogating the concentrate in the microstructured surface includes detecting an enzyme released from a live cell in the sample.
Embodiment 25 is the method of any of embodiments 1-24, wherein interrogating the concentrate in the microstructured surface includes detecting the analyte of interest colorimetrically, fluorimetrically, luminetrically, or a combination thereof.
Embodiment 26 is the method of any of embodiments 1-25, wherein the microstructured surface includes a recess density of at least about 100 recesses per square centimeter.
Embodiment 27 is the method of any of embodiments 1-26, wherein the microstructured surface includes a recess density of at least about 800 recesses per square centimeter.
Embodiment 28 is the method of any of embodiments 1-27, wherein the microstructured surface includes a recess density of at least about 3000 recesses per square centimeter.
Embodiment 29 is the method of any of embodiments 1-28, wherein the microstructured surface comprises a plurality of recesses, and wherein each of the plurality of recesses contains a volume of no greater than 1 microliter.
Embodiment 30 is the method of any of embodiments 1-29, wherein the microstructured surface comprises a plurality of recesses, wherein the plurality of recesses define a collective volume, and wherein the collective volume is no greater than 100 microliters.
Embodiment 31 is the method of any of embodiments 1-30, wherein the microstructured surface comprises a plurality of recesses, and wherein at least one of the plurality of recesses comprises a reagent.
Embodiment 32 is the method of embodiment 31, wherein the reagent includes at least one of a substrate, an enzyme, a growth reagent, a lysis reagent, or a combination thereof.
Embodiment 33 is the method of any of embodiments 1-32, wherein the analyte of interest is detected in the concentrate in no greater than 8 hours, if the analyte is present in the sample.
Embodiment 34 is the method of any of embodiments 1-33, wherein the analyte of interest is detected in the concentrate in no greater than 3 hours, if the analyte is present in the sample.
Embodiment 35 is the method of any of embodiments 1-34, wherein optically interrogating includes directing electromagnetic energy toward the concentrate in the microstructured surface at a first frequency; detecting electromagnetic energy emitted from the concentrate in the microstructured surface at a second frequency; directing electromagnetic energy toward the concentrate in the microstructured surface at a third frequency; and detecting electromagnetic energy emitted from the concentrate in the microstructured surface at a fourth frequency.
Embodiment 36 is the method of embodiment 35, wherein the first frequency is the energy for excitation associated with the product from the reaction of the H₂S probe with H₂S and the third frequency is the energy for excitation associated with the product from the reaction of the enzyme substrate with an enzyme.
Embodiment 37 is the method of embodiment 35, wherein the second frequency is the emitted energy associated with the product from the reaction of the H₂S probe with H₂S and the fourth frequency is the emitted energy associated with the product from the reaction of the enzyme substrate with an enzyme.
Embodiment 38 is the method of embodiment 36 or 37, wherein the enzyme is a phosphatase, as protease, peroxidase or esterase.
Embodiment 39 is the method of any of embodiments 1-38, wherein the H₂S probe reacts with H₂S to form iron(II) sulfide.
Embodiment 40 is the method of any of embodiments 1-39, wherein the H₂S probe reacts with H₂S to form iron(II) sulfide as a black precipitate.
Embodiment 41 is the method of any of embodiments 1-40, wherein the H₂S probe reacts with H₂S to form a fluorescent product.
Embodiment 42 is the method of any of embodiments 1-41, wherein the H₂S probe acts as a colorimetric or fluorescent indicator.
Embodiment 43 is the method of any of embodiments 1-42, wherein the enzyme substrate acts as a fluorescent indicator.
Embodiment 44 is the method of any of embodiments 1-43, wherein the enzyme substrate is a substrate for phosphatase or esterase.
Embodiment 45 is the method of any of embodiments 1-44, wherein the enzyme substrate reacts with a phosphatase or esterase to form a fluorescent product.
Embodiment 46 is the method of any of embodiments 1-45, wherein the enzyme substrate is selected from the group consisting of MUP, DiFMUP, DiFMU, MU-Ac, FDA, FDP, CFDA, DFDA, RFA, MU-Bu, BzUA, 7-amino-4-methylcoumarin based substrates, and 7-amino-4-chloromethylcoumarin based substrates.
Embodiment 47 is the method of any of embodiments 1-46, wherein the enzyme substrate is selected from the group consisting of MUP, DiFMUP, MU-Ac, FDA, and CFDA.
Embodiment 48 is the method of any of embodiments 1-47, wherein the H₂S probe is selected from WSP-1 and AzMC.
Embodiment 49 is the method of any of embodiments 1-48, wherein the H₂S probe is WSP-1 and the enzyme substrate is selected from the group consisting of MUP, DiFMUP, DiFMU, MU-Ac, FDA, FDP, CFDA, DFDA, RFA, MU-Bu, BzUA, 7-amino-4-methylcoumarin based substrates, and 7-amino-4-chloromethylcoumarin based substrates.
Embodiment 50 is the method of any of embodiments 1-49, wherein the H₂S probe is WSP-1 and the enzyme substrate is selected from the group consisting of MUP, DiFMUP, MU-Ac, FDA, and CFDA.
Embodiment 51 is the method of any of embodiments 1-50, wherein the H₂S probe is AzMC and the enzyme substrate is selected from the group consisting of MUP, DiFMUP, DiFMU, MU-Ac, FDA, FDP, CFDA, DFDA, RFA, MU-Bu, BzUA, 7-amino-4-methylcoumarin based substrates, and 7-amino-4-chloromethylcoumarin based substrates.
Embodiment 52 is the method of any of embodiments 1-51, wherein the H₂S probe is AzMC and the enzyme substrate is selected from the group consisting of MUP, DiFMUP, MU-Ac, FDA, and CFDA.
Embodiment 53 is the method of any of embodiments 1-52, wherein the sample is a water sample.
Embodiment 54 is the method of any of embodiments 1-53, wherein the sample is an oil-field or gas-field water sample.
Embodiment 55 is the method of any of embodiments 1-54, wherein the sample is an oil field water sample.
Embodiment 56 is the method of any of embodiments 1-55, wherein the analyte is selected for detecting the presence or absence of sulfate reducing bacteria.
Embodiment 57 is the method of embodiment 56, wherein the sulfate reducing bacteria is Desulfovibrio spp. or Desulfotomaculum spp.
Embodiment 58 is a method of detecting an analyte of interest comprising:

providing a container adapted to receive a sample, the container comprising a microstructured surface configured to provide capillary forces to retain a sample of interest;

positioning a sample in the container;

adding a probe and an enzyme substrate to the container;

centrifuging the container toward the microstructured surface to form a sediment and a supernatant of the sample;

inverting the container, after centrifuging the container, to remove at least a portion of the supernatant of the sample from being in contact with the microstructured surface, such that a concentrate of the sample is retained in the microstructured surface, the concentrate comprising the sediment; and

interrogating the concentrate in the microstructured surface for the analyte of interest.

Embodiment 59 is a method of detecting an analyte of interest comprising:

providing a container adapted to receive a sample, the container having a probe and an enzyme substrate, wherein the container comprises a microstructured surface;

positioning a sample in the container;

centrifuging the container toward the microstructured surface to form a sediment and a supernatant of the sample;

inverting the container, after centrifuging the container, to remove at least a portion of the supernatant of the sample from being in contact with the microstructured surface, such that a concentrate of the sample is retained in the microstructured surface, the concentrate comprising the sediment; and

interrogating the concentrate in the microstructured surface for the analyte of interest.

Embodiment 60 is a method of detecting an analyte of interest comprising:

providing a container adapted to receive a sample, the container having a probe and an enzyme substrate and the container comprising an open end configured to receive a sample and a closed end, the closed end comprising:
a first side comprising a microstructured surface, the first side facing an interior of the container, and

a second side opposite the first side and facing outside of the container, wherein at least a portion of the container is substantially transparent such that the microstructured surface is visible from the second side;

flushing the container with an inert gas;

positioning a sample in the container;

centrifuging the container toward the microstructured surface to form a sediment and a supernatant of the sample;

inverting the container, after centrifuging the container, to remove at least a portion of the supernatant of the sample from being in contact with the microstructured surface, such that a concentrate of the sample is retained in the microstructured surface, the concentrate comprising the sediment; and

interrogating the concentrate in the microstructured surface for the analyte of interest, wherein interrogating the concentrate in the microstructured surface includes interrogating the concentrate from the second side of the container.

Embodiment 61 is a method of detecting an analyte of interest comprising:

providing a container adapted to receive a sample, the container comprising an open end configured to receive a sample and a closed end, the closed end comprising:

a first side comprising a microstructured surface, the first side facing an interior of the container, and

a second side opposite the first side and facing outside of the container, wherein at least a portion of the container is substantially transparent such that the microstructured surface is visible from the second side;

flushing the container with an inert gas;

positioning a sample in the container;

adding an H₂S probe and an enzyme substrate to the container;

centrifuging the container toward the microstructured surface to form a sediment and a supernatant of the sample;

inverting the container, after centrifuging the container, to remove at least a portion of the supernatant of the sample from being in contact with the microstructured surface, such that a concentrate of the sample is retained in the microstructured surface, the concentrate comprising the sediment; and

interrogating the concentrate in the microstructured surface for the analyte of interest, wherein interrogating the concentrate in the microstructured surface includes interrogating the concentrate from the second side of the container.

Embodiment 62 is the method of any of embodiments 1-61, wherein the microstructured surface is configured to provide capillary forces to retain the concentrate of the sample.
Embodiment 63 is the method of any of embodiments 1-62, wherein the open end is sealed.
Embodiment 64 is the method of any of embodiments 1-63, wherein the open end is sealed with a septum.
Embodiment 65 is the method of any of embodiments 1-63, wherein interrogating the concentrate in the microstructured surface includes interrogating the concentrate from the second side.
Embodiment 66 is the method of embodiment, wherein 35 wherein the first frequency is the energy for excitation associated with the product from the reaction of the H₂S probe with H₂S and for excitation associated with the product from the reaction of the enzyme substrate with an enzyme.
Embodiment 67 is an article comprising:

a container adapted to receive a sample, the container comprising an open end configured to receive a sample and a closed end, the closed end comprising:
a first side comprising a microstructured surface, the first side facing an interior of the container, and
a second side opposite the first side and facing outside of the container, wherein at least a portion of the container is substantially transparent such that the microstructured surface is visible from the second side;

a probe and an enzyme substrate disposed in the container.

Embodiment 68 is the article of embodiment 67, wherein the probe is an H₂S probe.

The following working and prophetic examples are intended to be illustrative of the present disclosure and not limiting.

### EXAMPLES

### Materials and Instruments

Transparent cyclic olefin copolymer (tCOC), high moisture barrier (TOPAS 8007S-04), was obtained from TOPAS Advanced Polymers Gmbh, Florence, Ky.

LEXAN HPH4404, a high heat specialty polycarbonate (ethylene oxide, steam, gamma and e-beam sterilizable), was obtained from SABIC Innovative Plastics, Pittsfield, Mass.

A multipurpose centrifuge (Model 5804) with a swinging bucket rotor was obtained from Eppendorf, Hauppauge, N.Y.

The imaging system was an illuminated/fluorescent stereo microscope model SteREO Lumar.V12 that used a fluorescence-Hg lamp with excitation and emission filter sets for UV, blue, green, and yellow. Images were captured with an AxioCam MRc 5 camera and the AxioVision Release 4.6.3 program. All obtained from Carl Zeiss Microimaging, Inc., Thornwood, N.J. The microstructured surface of each container was imaged from the exterior of the container.

mSLS medium (modified sodium lactate for sulfate reducers medium without ammonium iron(II) sulfate) [composition: yeast extract 1 g/L, MgSO₄.7H₂O 1 g/L, NH₄Cl 0.4 g/L, K₂HPO₄ 0.01 g/L, NaCl 5 g/L, sodium ascorbate 0.1 g/L, and sodium lactate (60%) 4 mL/L] was prepared according to the NACE TM0194-2004 standard test method (Nace International, Houston, Tex.). The medium was prepared, adjusted to pH 7.3 with NaOH, de-aerated with nitrogen, and sterilized in an autoclave at 121° C. for 15 min. The medium (10 mL) was then dispensed into a glass anaerobic tube (18×150 mm with a 20 mm blue chlorobutyl rubber stopper and crimped aluminum seal, cat. # CLS-4209-01, Chemglass Life Sciences, Vineland, N.J.).

Washington State Probe-1 (WSP-1, 3′-methoxy-3-oxo-3H-spiro[isobenzofuran-1,9′-xanthen]-6′-yl 2-(pyridin-2-yldisulfanyl)benzoate; obtained from Cayman Chemicals, Ann Arbor, Mich.) and AzMC (7-azido-4-methylcoumarin; obtained from Sigma-Aldrich Corporation, St. Louis, Mo.), were used as fluorescent probes to detect H₂S in the examples.

The fluorescent enzyme substrates 4-methylumbelliferyl phosphate (MUP), 6,8-difluoro-4-methylumbelliferyl phosphate (DiFMUP), 4-methylumbelliferyl acetate (MU-Ac), fluorescein diacetate (FDA), and 5(6)-carboxyfluorescein diacetate (CFDA) were obtained from Thermo Fisher Scientific, Waltham, Mass.).

TABLE 1
Excitation and Emission Wavelengths for
H₂S Probes and Enzyme Substrates

| | Excitation | Emission |
|---|---|---|
| | Wavelength | Wavelength |
| Enzyme Substrates | | |
| MUP | 365 nm | 450 nm |
| DiFMUP | 365 nm | 450 nm |
| MU-Ac | 365 nm | 450 nm |
| CFDA | 485 nm | 520 nm |
| FDA | 485 nm | 520 nm |
| H₂S Probes | | |
| WSP-1 | 485 nm | 520 nm |
| AzMC | 365 nm | 450 nm |

TABLE 2
Excitation and Emission Wavelengths for H₂S
Probe and Enzyme Substrate Combinations

| | H₂S Probe | | Enzyme Substrate | |
|---|---|---|---|---|
| | Excitation | Emission | Excitation | Emission |
| Combination | Wavelength | Wavelength | Wavelength | Wavelength |
| WSP-1 + MUP | 485 nm | 520 nm | 365 nm | 450 nm |
| WSP-1 + | 485 nm | 520 nm | 365 nm | 450 nm |
| DiFMUP | | | | |
| WSP-1 + | 485 nm | 520 nm | 365 nm | 450 nm |
| MU-Ac | | | | |
| AzMC + | 365 nm | 450 nm | 485 nm | 520 nm |
| CFDA | | | | |
| AzMC + FDA | 365 nm | 450 nm | 485 nm | 520 nm |

### Example 1. Preparation of Containers with a Molded Microstructure Surface

Substantially transparent containers (FIG. 1) with a molded microstructured surface (15 ml capacity) were injection molded in a KraussMaffei injection molding machine (Model K65-CX, KraussMaffei technologies, Munich, Germany) with transparent cyclic olefin copolymer (tCOC) resin (TOPAS 8007S-04) or polycarbonate resin (LEXAN HPH4404). The resin pellets for TOPAS 8007S-04 were melted at 232 to 238° C., and then injected at 16,000 psi. The mold temperature was held at 66° C. and the injection time was 0.78 sec. The resin pellets for Lexan HPH4004 were melted at 270 to 300° C., and then injected at 26,000 psi. The mold temperature was held at 85 to 90° C. and the injection time was 0.59 sec. Each part was made individually during molding.

Each molded container was cylindrical in shape with a flat closed end and an opposite open end (outer diameter=24 mm, height=47 mm). The microstructured surface was molded into the internal surface of the closed end of the container as a frustum of pyramid microstructures (which were in the form of recesses or wells). The steel template for the microstructured surface was made using tooling techniques such as electrical discharge machining (EDM), wire-EDM, and polishing to form the inverse of the desired features in the template. The dimensions for the microstructured surface of the containers are provided in Table 3. Each well is characterized by a two-dimensional (e.g., cross-sectional) shape having a top opening, one or more sidewalls and a bottom. The draft angle was calculated as the angle formed by a line perpendicular to the bottom of the well and a sidewall of the well. The volume (nanoliters, nL) of each well was defined by the area of the top and bottom, measured as the distance in microns through the center point from one edge to the opposite edge and the depth—the distance in microns from the top of the well to the bottom of the well. The aspect ratio was calculated as the depth of a well divided by the side dimension for the top of the well. Pitch was measured as the center to center between adjacent wells. The wells of the microstructured surface faced the interior of the container (i.e. the internal surfaces of the wells were oriented to be able to be in contact with a fluid added to the container).

TABLE 3
Physical Dimensions of Microstructured Wells

| Density (Number | | | Top | Bottom | Well | Calculated | Volume | Aspect |
|---|---|---|---|---|---|---|---|---|
| of Wells | Well | Pitch | Side | Side | Depth | draft Angle | of Well | Ratio of |
| per cm²) | Shape | (□m) | (□m) | (□m) | (□m) | (degrees) | (nL) | Well |
| 826 | Square | 348 | 228 | 100 | 254 | 14 | 7 | 1.11 |

The edge of the open end of the molded container contained an extended lip portion (width=3 mm) that received a stopper (gray bromobutyl rubber, snap-on type stopper, 30 mm diameter, catalog # W224100-342, Wheaton, Millville, N.J.). A plastic, circular spacer with a centered hollow bore (bore diameter=8 mm, spacer outer diameter=28 mm, spacer thickness=3.5 mm) was placed on top of the inserted stopper. An aluminum cap seal (30 mm diameter with a center tear seal, catalog #224187-01, Wheaton) was placed over the stopper and spacer and crimped to cap and seal the open end of the container.

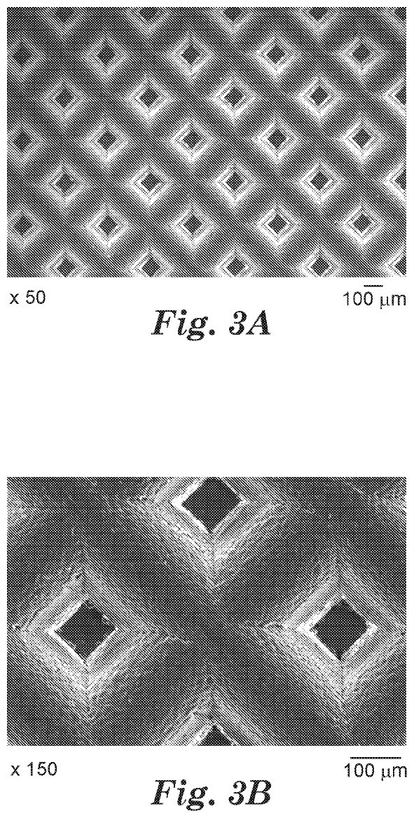

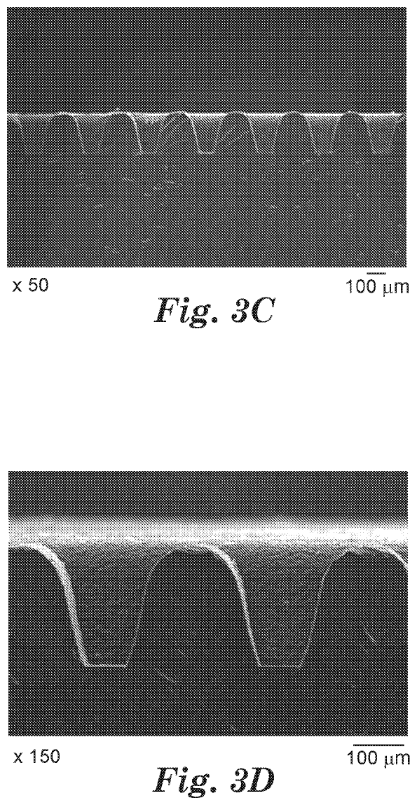

Scanning electron microscopy (SEM) images of the microstructured wells of the container were taken at magnifications of 50× and 150×. Samples for surface imaging were prepared by cutting the microstructure region from the container. The samples were then mounted on an aluminum stub and sputter coated with gold/palladium. The resulting coated samples were then examined using a JSM-7001F Scanning Electron Microscope (JEOL Ltd, Tokyo, Japan). Surface images were taken at a viewing angle of 70° off the surface of the stub. Additional samples were prepared for cross-section imaging by submerging a sample in liquid nitrogen and striking the sample with a hammer. A cross sections fragment was mounted on an aluminum stub, sputter coated, and examined with the scanning electron microscope. The cross section images were taken at viewing angle normal to the surface of the sectioned face. Optical images of microstructured wells are shown in FIGS. 3A-3D.

### Example 2. Preparation of Bacterial Cultures

SRB cultures used in the examples were prepared from Desulfovibrio vulgaris (ATCC No. 29579) and Desulfovibrio desulfuricans (ATCC No. 29577) which were obtained from American Tissue Culture Collection (ATCC), Manassas, Va.).

Stock cultures were grown using modified Postgate B medium without ferrous sulfate [composition: KH₂PO₄ 0.5 g/L, NH₄Cl 1.0 g/L, CaSO₄ 1.0 g/L, MgSO₄ 7H₂O 2.0 g/L, sodium lactate 50% 5.5 mL/L, yeast extract 1.0 g/L, ascorbic acid 0.1 g/L, thioglycollic acid 80% 0.1 mL/L] according to the NACE TM0194-2004 standard test method (NACE International) and stored at 4° C. for up to a week. The media was prepared, de-aerated with nitrogen, sterilized by autoclaving, dispensed into glass tubes under nitrogen, closed with a butyl rubber stopper and sealed by crimping with an aluminum seal. The tubes were also flushed with nitrogen to remove any traces of oxygen and pressurized to about 10 psi. For serial dilutions (10-fold) of the bacteria, syringes with a 22 gauge needle were first flushed with nitrogen and the dilutions were prepared using the Postgate B media. A known dilution was used to inoculate the containers.

### Example 3. Detection of SRB Using an H₂S Probe (Formation of Iron(II) Sulfide Precipitate)

Containers (tCOC and PC Lexan) with a molded microstructured surface (capped and sealed as described above) were flushed with nitrogen for about 5 minutes and then pressurized to 10 psi with nitrogen. Sulfate reducing bacteria media (10 mL) (obtained from an INTERTEK MIC Test Kit cat #08-629-008A, Thermo Fisher Scientific, Waltham, Mass.) was withdrawn from an INTERTEK vial (clear glass serum vial) using a syringe (flushed with nitrogen) and added aseptically to each container. A 0.1 ml aliquot of either the D. vulgaris or D. desulfuricans suspension culture (approximately 100 cfu) was then added aseptically to each container. The containers were centrifuged for 15 minutes at 5000 rpm. The containers were removed from the centrifuge, slowly inverted to decant the bulk of the media away from the microstructured surface, and then incubated at 30° C. The containers were maintained in the inverted position throughout the remainder of the experiment (i.e. during both incubation and detection).

At the same time, INTERTEK serum vials containing Intertek sulfate reducing bacteria media (from the test kit described above) were also aseptically inoculated with 0.1 ml aliquots of either the D. vulgaris or D. desulfuricans suspension culture (approximately 100 cfu). The INTERTEK serum vials served as Comparative Example 1. The containers and vials were incubated at 30° C. and evaluated for the appearance of a black precipitate (iron(II) sulfide, FeS) after 8, 16, 24, 48, and 72 hours of incubation. The microstructured surfaces of the inverted containers were imaged using the stereo microscope imager system (described above) with a white light. The INTERTEK vials (Comparative Example 1) were evaluated for a black precipitate by visual examination. A total of 3 replicates were evaluated for each container/vial and SRB combination. A black precipitate was visualized in the microstructures of both tCOC and Lexan containers at 24 hours of incubation. However, a black precipitate was not detected in the INTERTEK vials (Comparative Example 1) until 72 hours of incubation. The results are summarized in Table 4.

TABLE 4
Comparison of time to detect SRB

| | Black Precipitate Detected | | | |
|---|---|---|---|---|
| | D. vulgaris | | D. desulfuricans | |
| | | Container | | Container |
| | INTERTEK | with | INTERTEK | with |
| | Vial | Micro- | Vial | Micro- |
| | (Comparative | structured | (Comparative | structured |
| Time | Example 1) | Surface | Example 1) | Surface |
| 8 hr | No | No | No | No |
| 16 hr | No | No | No | No |
| 24 hr | No | Yes | No | Yes |
| 48 hr | No | Yes | No | Yes |
| 72 hr | Yes | Yes | Yes | Yes |

### Example 4. Detection of SRB Using a Fluorescent H₂S Probe

Containers (tCOC and PC Lexan) with a molded microstructured surface (capped and sealed as described above) were flushed with nitrogen for about 5 minutes and then pressurized to 10 psi with nitrogen. mSLS media (10 mL) was withdrawn from an anaerobic storage tube using a syringe (flushed with nitrogen) and added aseptically to each container. Either WSP-1 (1 mg/mL solution in DMSO) or AzMC (1 mg/mL solution in DMSO) was then added aseptically to achieve a 10 micromolar concentration of the H₂S probe in the mSLS. A 0.1 ml aliquot of either the D. vulgaris or D. desulfuricans suspension culture (approximately 100 cfu) was then added aseptically to each container. The containers were centrifuged for 15 minutes at 5000 rpm. The containers were removed from the centrifuge, slowly inverted to decant the bulk of the media away from the microstructured surface, and then incubated at 30° C. The containers were maintained in the inverted position throughout the remainder of the experiment (i.e. during both incubation and detection).

At the same time, anaerobic tubes containing 10 mL of mSLS medium (preparation described above) were prepared in a similar manner to serve as Comparative Example 2. To each anaerobic tube either WSP-1 (1 mg/mL solution in DMSO) or AzMC (1 mg/mL solution in DMSO) was added aseptically to achieve a 10 micromolar concentration of the H₂S probe in the mSLS. A 0.1 ml aliquot of either the D. vulgaris or D. desulfuricans suspension culture (approximately 100 cfu) was then added aseptically to each tube and the tubes were incubated at 30° C.

The containers and anaerobic tubes were evaluated for a fluorescence signal at 3, 6, 8, 12, 16, 20, and 24 hours of incubation. The detection of a fluorescence signal indicated the presence of the SRB (D. vulgaris or D. desulfuricans). The microstructured surfaces of the inverted containers were imaged using the stereo microscope imager system (described above) and the excitation and emission wavelengths listed in Table 1. The anaerobic tubes (Comparative Example 2) were evaluated using the same imaging system. A total of 3 replicates were evaluated for each container/tube/H₂S probe and SRB combination. A fluorescence signal was visualized in the microstructures of both tCOC and Lexan containers at 8 hours of incubation. However, a fluorescence signal was not detected in the anaerobic tubes (Comparative Example 2) until 24 hours of incubation. The results are summarized in Tables 5 and 6.

TABLE 5
Comparison of time to detect SRB (with H₂S Probe)

| | Detection of Fluorescence (WSP-1 as the H₂S Probe) | | | |
|---|---|---|---|---|
| | | | D. desulfuricans | |
| | D. vulgaris | | | Container |
| | Anaerobic tube | Container with | Anaerobic tube | with Micro- |
| | (Comparative | Microstructured | (Comparative | structured |
| Time | Example 2) | Surface | Example 2) | Surface |
| 3 hr | No | No | No | No |
| 6 hr | No | No | No | No |
| 8 hr | No | Yes | No | Yes |
| 12 hr | No | Yes | No | Yes |
| 16 hr | No | Yes | No | Yes |
| 20 hr | No | Yes | No | Yes |
| 24 hr | Yes | Yes | Yes | Yes |

TABLE 6
Comparison of time to detect SRB (with H₂S Probe)

| | Detection of Fluorescence (AzMC as the H₂S Probe) | | | |
|---|---|---|---|---|
| | | | D. desulfitricans | |
| | D. vulgaris | | | Container |
| | Anaerobic tube | Container with | Anaerobic tube | with Micro- |
| | (Comparative | Microstructured | (Comparative | structured |
| Time | Example 2) | Surface | Example 2) | Surface |
| 3 hr | No | No | No | No |
| 6 hr | No | No | No | No |
| 8 hr | No | Yes | No | Yes |
| 12 hr | No | Yes | No | Yes |
| 16 hr | No | Yes | No | Yes |
| 20 hr | No | Yes | No | Yes |
| 24 hr | Yes | Yes | Yes | Yes |

### Example 5. Detection of SRB Using an Enzyme Substrate

Individual solutions of the five enzyme substrates MUP, DiFMUP, MU-Ac, FDA, and CFDA were prepared by dissolving each enzyme substrate in a separate vial of DMSO at a concentration of 1 mg/mL. Containers (tCOC and PC Lexan) with a molded microstructured surface (capped and sealed as described above) were flushed with nitrogen for about 5 minutes and then pressurized to 10 psi with nitrogen. mSLS media (10 mL) was withdrawn from an anaerobic storage tube using a syringe (flushed with nitrogen) and added aseptically to each container. One of the enzyme substrate solutions was then added aseptically to achieve a 10 micromolar concentration of the enzyme substrate in the mSLS. A 0.1 ml aliquot of either the D. vulgaris or D. desulfuricans suspension culture (approximately 100 cfu) was then added aseptically to each container. The containers were centrifuged for 15 minutes at 5000 rpm. The containers were removed from the centrifuge, slowly inverted to decant the bulk of the media away from the microstructured surface, and then incubated at 30° C. The containers were maintained in the inverted position throughout the remainder of the experiment (i.e. during both incubation and detection).

At the same time, anaerobic tubes containing 10 mL of mSLS medium (preparation described above) were prepared in a similar manner to serve as Comparative Example 3. To each anaerobic tube a solution (1 mg/mL in DMSO) of either MUP, DiFMUP, FDA, or CFDA was added aseptically to achieve a 10 micromolar concentration of the enzyme substrate in the mSLS. A 0.1 ml aliquot of either the D. vulgaris or D. desulfuricans suspension culture (approximately 100 cfu) was then added aseptically to each tube and the tubes were incubated at 30° C.

The containers and anaerobic tubes were evaluated for a fluorescence signal at 3, 6, 8, 12, 16, 20, and 24 hours of incubation. The detection of a fluorescence signal indicated the presence of the SRB (D. vulgaris or D. desulfuricans). The microstructured surfaces of the inverted containers were imaged using the stereo microscope imager system (described above) and the excitation and emission wavelengths listed in Table 1. The anaerobic tubes (Comparative Example 3) were evaluated using the same imaging system. A total of 3 replicates were evaluated for each container/tube/enzyme substrate and SRB combination. For all of the enzyme substrates, a fluorescence signal was visualized in the microstructures of both tCOC and Lexan containers at 8 hours of incubation. However, a fluorescence signal was not detected in the anaerobic tubes (Comparative Example 3) until 24 hours of incubation. The results are summarized in Tables 7 and 8 for the microstructured containers and in Tables 9 and 10 for the anaerobic tubes (Comparative Example 3).

TABLE 7
Time to detect Fluorescence from Enzyme Substrates Using
a Container with a Microstructured Surface

| | Detection of Fluorescence with Enzyme Substrate | | | | |
|---|---|---|---|---|---|
| | (Microstructured Surface) | | | | |
| | D. vulgaris | | | | |
| Time | MUP | DiFMUP | MU-Ac | FDA | CFDA |
| 3 hr | No | No | No | No | No |
| 6 hr | No | No | No | No | No |
| 8 hr | Yes | Yes | Yes | Yes | Yes |
| 12 hr | Yes | Yes | Yes | Yes | Yes |
| 16 hr | Yes | Yes | Yes | Yes | Yes |
| 20 hr | Yes | Yes | Yes | Yes | Yes |
| 24 hr | Yes | Yes | Yes | Yes | Yes |

TABLE 8
Time to detect Fluorescence from Enzyme Substrates Using
a Container with a Microstructured Surface

| | Detection of Fluorescence with Enzyme Substrate | | | | |
|---|---|---|---|---|---|
| | (Microstructured Surface) | | | | |
| | D. desulfuricans | | | | |
| Time | MUP | DiFMUP | MU-Ac | FDA | CFDA |
| 3 hr | No | No | No | No | No |
| 6 hr | No | No | No | No | No |
| 8 hr | Yes | Yes | Yes | Yes | Yes |
| 12 hr | Yes | Yes | Yes | Yes | Yes |
| 16 hr | Yes | Yes | Yes | Yes | Yes |
| 20 hr | Yes | Yes | Yes | Yes | Yes |
| 24 hr | Yes | Yes | Yes | Yes | Yes |

TABLE 9
Time to detect Fluorescence from Enzyme Substrates
Using an Anaerobic Tube (Comparative Example 3)

| | Detection of Fluoresence with Enzyme | | | | |
|---|---|---|---|---|---|
| | Substrate (using Anaerobic Tube) | | | | |
| | D. vulgaris | | | | |
| Time | MUP | DiFMUP | MU-Ac | FDA | CFDA |
| 3 hr | No | No | No | No | No |
| 6 hr | No | No | No | No | No |
| 8 hr | No | No | No | No | No |
| 12 hr | No | No | No | No | No |
| 16 hr | No | No | No | No | No |
| 20 hr | No | No | No | No | No |
| 24 hr | Yes | Yes | Yes | Yes | Yes |

TABLE 10
Time to detect Fluorescence from Enzyme Substrates
Using an Anaerobic Tube (Comparative Example 3)

| | Detection of Fluoresence with Enzyme | | | | |
|---|---|---|---|---|---|
| | Substrate (using Anaerobic Tube) | | | | |
| | D. desulfuricans | | | | |
| Time | MUP | DiFMUP | MU-Ac | FDA | CFDA |
| 3 hr | No | No | No | No | No |
| 6 hr | No | No | No | No | No |
| 8 hr | No | No | No | No | No |
| 12 hr | No | No | No | No | No |
| 16 hr | No | No | No | No | No |
| 20 hr | No | No | No | No | No |
| 24 hr | Yes | Yes | Yes | Yes | Yes |

### Example 6. Detection of SRB Using a Hydrogen Sulfide Probe and an Enzyme Substrate

Individual solutions of the three enzyme substrates MUP, DiFMUP, and MU-Ac, were prepared by dissolving each enzyme substrate in a separate vial of DMSO at a concentration of 1 mg/mL. Containers (tCOC and PC Lexan) with a molded microstructured surface (capped and sealed as described above) were flushed with nitrogen for about 5 minutes and then pressurized to 10 psi with nitrogen. mSLS media (10 mL) was withdrawn from an anaerobic storage tube using a syringe (flushed with nitrogen) and added aseptically to each container. To each anaerobic tube WSP-1 (1 mg/mL solution in DMSO) was added aseptically to achieve a 10 micromolar concentration of the H₂S probe in the mSLS.

One of the enzyme substrate solutions was then added aseptically to achieve a 10 micromolar concentration of the enzyme substrate in the mSLS. A 0.1 ml aliquot of either the D. vulgaris suspension culture (approximately 100 cfu) was then added aseptically to each container. The containers were centrifuged for 15 minutes at 5000 rpm. The containers were removed from the centrifuge, slowly inverted to decant the bulk of the media away from the microstructured surface, and then incubated at 30° C. The containers were maintained in the inverted position throughout the remainder of the experiment (i.e. during both incubation and detection).

At the same time, anaerobic tubes containing 10 mL of mSLS medium (preparation described above) were prepared in a similar manner to serve as Comparative Example 4. To each anaerobic tube WSP-1 (1 mg/mL solution in DMSO) was added aseptically to achieve a 10 micromolar concentration of the H₂S probe in the mSLS. Next, to each anaerobic tube a solution (1 mg/mL in DMSO) of either MUP, DiFMUP, or MU-AC was added aseptically to achieve a 10 micromolar concentration of the enzyme substrate in the mSLS. A 0.1 ml aliquot of D. vulgaris suspension culture (approximately 100 cfu) was then added aseptically to each tube and the tubes were incubated at 30° C.

The containers and anaerobic tubes were evaluated for a fluorescence signal at 3, 6, 8, 12, 16, 20, and 24 hours of incubation. The detection of a fluorescence signal indicated the presence of the SRB. The microstructured surfaces of the inverted containers were imaged using the stereo microscope imager system (described above) and the appropriate excitation and emission filter set for each indicator (i.e the surface was imaged first using the appropriate excitation/emission filter set for detecting fluorescence from the WSP-1 probe and then imaged a second time using the appropriate excitation/emission filter set for detecting fluorescence from the enzyme substrate. The appropriate excitation and emission wavelengths are listed in Table 2. The anaerobic tubes (Comparative Example 4) were evaluated using the same imaging system. A total of 3 replicates were evaluated for each container/tube/H₂S probe/enzyme substrate combination. For all containers with microstructured surfaces (tCOC and Lexan) fluorescence signals from both the H₂S probe (WSP-1) and the enzyme substrate (MUP, DiFMUP, or MU-Ac) were detected after 8 hours of incubation. However, the corresponding fluorescence signals were not detected in the anaerobic tubes (Comparative Example 4) until 24 hours of incubation. The results are summarized in Table 11 for the microstructured containers and in Table 12 for the anaerobic tubes (Comparative Example 4).

TABLE 11
Time to detect D. vulgaris using both an H₂S Probe and an Enzyme
Substrate in a Container with a Microstructured Surface

| | | Detection of Individual Fluorescence Signals Using | | | | | |
|---|---|---|---|---|---|---|---|
| | | Both an H₂S Probe and Enzyme Substrate | | | | | |
| | | WSP-1 + MUP | | WSP-1 + DiFMUP | | WSP-1 + MU-Ac | |
| | | Signal | Signal | Signal | Signal | Signal | Signal |
| | | from | from | from | from | from | from |
| Time | | WSP-1 | MUP | WSP-1 | DiFMUP | WSP-1 | MU-Ac |
| 3 | hr | No | No | No | No | No | No |
| 6 | hr | No | No | No | No | No | No |
| 8 | hr | Yes | Yes | Yes | Yes | Yes | Yes |
| 12 | hr | Yes | Yes | Yes | Yes | Yes | Yes |
| 16 | hr | Yes | Yes | Yes | Yes | Yes | Yes |
| 20 | hr | Yes | Yes | Yes | Yes | Yes | Yes |
| 24 | hr | Yes | Yes | Yes | Yes | Yes | Yes |

TABLE 12
Time to detect D. vulgaris using both an H₂S Probe and an
Enzyme Substrate in an Anaerobic Tube (Comparative Example 4)

| | | Detection of Individual Fluorescence Signals Using | | | | | |
|---|---|---|---|---|---|---|---|
| | | Both an H₂S Probe and Enzyme Substrate | | | | | |
| | | WSP-1 + MUP | | WSP-1 + DiFMUP | | WSP-1 + MU-Ac | |
| | | Signal | Signal | Signal | Signal | Signal | Signal |
| | | from | from | from | from | from | from |
| Time | | WSP-1 | MUP | WSP-1 | DiFMUP | WSP-1 | MU-Ac |
| 3 | hr | No | No | No | No | No | No |
| 6 | hr | No | No | No | No | No | No |
| 8 | hr | No | No | No | No | No | No |
| 12 | hr | No | No | No | No | No | No |
| 16 | hr | No | No | No | No | No | No |
| 20 | hr | No | No | No | No | No | No |
| 24 | hr | Yes | Yes | Yes | Yes | Yes | Yes |

### Example 7. Detection of SRB Using a Hydrogen Sulfide Probe and an Enzyme Substrate

Individual solutions of the two enzyme substrates FDA and CFDA were prepared by dissolving each enzyme substrate in a separate vial of DMSO at a concentration of 1 mg/mL. Containers (tCOC and PC Lexan) with a molded microstructured surface (capped and sealed as described above) were flushed with nitrogen for about 5 minutes and then pressurized to 10 psi with nitrogen. mSLS media (10 mL) was withdrawn from an anaerobic storage tube using a syringe (flushed with nitrogen) and added aseptically to each container. To each anaerobic tube AzMC (1 mg/mL solution in DMSO) was added aseptically to achieve a 10 micromolar concentration of the H₂S probe in the mSLS. One of the enzyme substrate solutions was then added aseptically to achieve a 10 micromolar concentration of the enzyme substrate in the mSLS. A 0.1 ml aliquot of either the D. vulgaris suspension culture (approximately 100 cfu) was then added aseptically to each container. The containers were centrifuged for 15 minutes at 5000 rpm. The containers were removed from the centrifuge, slowly inverted to decant the bulk of the media away from the microstructured surface, and then incubated at 30° C. The containers were maintained in the inverted position throughout the remainder of the experiment (i.e. during both incubation and detection).

At the same time, anaerobic tubes containing 10 mL of mSLS medium (preparation described above) were prepared in a similar manner to serve as Comparative Example 5. To each anaerobic tube AzMC (1 mg/mL solution in DMSO) was added aseptically to achieve a 10 micromolar concentration of the H₂S probe in the mSLS. Next, to each anaerobic tube a solution (1 mg/mL in DMSO) of either FDA or CFDA was added aseptically to achieve a 10 micromolar concentration of the enzyme substrate in the mSLS. A 0.1 ml aliquot of D. vulgaris suspension culture (approximately 100 cfu) was then added aseptically to each tube and the tubes were incubated at 30° C.

The containers and anaerobic tubes were evaluated for a fluorescence signal at 3, 6, 8, 12, 16, 20, and 24 hours of incubation. The detection of a fluorescence signal indicated the presence of the SRB. The microstructured surfaces of the inverted containers were imaged using the stereo microscope imager system (described above) and the appropriate excitation and emission filter set for each indicator (i.e the surface was imaged first using the appropriate excitation/emission filter set for detecting fluorescence from the AzMC probe and then imaged a second time using the appropriate excitation/emission filter set for detecting fluorescence from the enzyme substrate). The appropriate excitation and emission wavelengths are listed in Table 2. The anaerobic tubes (Comparative Example 5) were evaluated using the same imaging system. A total of 3 replicates were evaluated for each container/tube/H₂S probe/enzyme substrate combination. For all containers with microstructured surfaces (tCOC and Lexan) fluorescence signals from both the H₂S probe (AzMC) and the enzyme substrate (FDA or CFDA) were detected after 8 hours of incubation. However, the corresponding fluorescence signals were not detected in the anaerobic tubes (Comparative Example 5) until 24 hours of incubation. The results are summarized in Table 13 for the microstructured containers and in Table 14 for the anaerobic tubes (Comparative Example 5).

TABLE 13
Time to detect D. vulgaris using Both an H₂S Probe and
an Enzyme Substrate in a Container with a Microstructured Surface

| | Detection of Individual Fluorescence Signals Using | | | |
|---|---|---|---|---|
| | Both an H₂S Probe and Enzyme Substrate | | | |
| | AzMC + FDA | | AzMC + CFDA | |
| | Signal from | Signal from | Signal from | Signal from |
| Time | AzMC | FDA | AzMC | CFDA |
| 3 hr | No | No | No | No |
| 6 hr | No | No | No | No |
| 8 hr | Yes | Yes | Yes | Yes |
| 12 hr | Yes | Yes | Yes | Yes |
| 16 hr | Yes | Yes | Yes | Yes |
| 20 hr | Yes | Yes | Yes | Yes |
| 24 hr | Yes | Yes | Yes | Yes |

TABLE 14
Time to detect D. vulgaris using both an H₂S Probe and an
Enzyme Substrate in an Anaerobic Tube (Comparative Example 5)

| | Detection of Individual Fluorescence Signals Using | | | |
|---|---|---|---|---|
| | Both an H₂S Probe and Enzyme Substrate | | | |
| | AzMC + FDA | | AzMC + CFDA | |
| | Signal from | Signal from | Signal from | Signal from |
| Time | AzMC | FDA | AzMC | CFDA |
| 3 hr | No | No | No | No |
| 6 hr | No | No | No | No |
| 8 hr | No | No | No | No |
| 12 hr | No | No | No | No |
| 16 hr | No | No | No | No |
| 20 hr | No | No | No | No |
| 24 hr | Yes | Yes | Yes | Yes |

### Example 8

The same procedure as reported for Example 6 was followed with the only exception being that D. vulgaris was replaced with D. desulfuricans. For all containers with microstructured surfaces (tCOC and Lexan) fluorescence signals from both the H₂S probe (WSP-1) and the enzyme substrate (MUP, DiFMUP or MU-Ac) were detected after 8 hours of incubation. However, the corresponding fluorescence signals were not detected in the anaerobic tubes (Comparative Example 6) after 20 hours of incubation. The results are summarized in Table 15 for the microstructured container and in Table 16 for the corresponding anaerobic tube (Comparative Example 6).

TABLE 15
Time to detect D. desulfuricans using both an H₂S Probe and an
Enzyme Substrate in a Container with a Microstructured Surface

| | | Detection of Individual Fluorescence Signals Using | | | | | |
|---|---|---|---|---|---|---|---|
| | | Both an H₂S Probe and Enzyme Substrate | | | | | |
| | | WSP-1 + MUP | | WSP-1 + DiFMUP | | WSP-1 + MU-Ac | |
| | | Signal | Signal | Signal | Signal | Signal | Signal |
| | | from | from | from | from | from | from |
| Time | | WSP-1 | MUP | WSP-1 | DiFMUP | WSP-1 | MU-Ac |
| 3 | hr | No | No | No | No | No | No |
| 6 | hr | No | No | No | No | No | No |
| 8 | hr | Yes | Yes | Yes | Yes | Yes | Yes |
| 12 | hr | Yes | Yes | Yes | Yes | Yes | Yes |
| 16 | hr | Yes | Yes | Yes | Yes | Yes | Yes |
| 20 | hr | Yes | Yes | Yes | Yes | Yes | Yes |

TABLE 16
Time to detect D. desulfuricans using both an H₂S Probe and an
Enzyme Substrate in an Anaerobic Tube (Comparative Example 6)

| | | Detection of Individual Fluorescence Signals Using | | | | | |
|---|---|---|---|---|---|---|---|
| | | Both an H₂S Probe and Enzyme Substrate | | | | | |
| | | WSP-1 + MUP | | WSP-1 + DiFMUP | | WSP-1 + MU-Ac | |
| | | Signal | Signal | Signal | Signal | Signal | Signal |
| | | from | from | from | from | from | from |
| Time | | WSP-1 | MUP | WSP-1 | DiFMUP | WSP-1 | MU-Ac |
| 3 | hr | No | No | No | No | No | No |
| 6 | hr | No | No | No | No | No | No |
| 8 | hr | No | No | No | No | No | No |
| 12 | hr | No | No | No | No | No | No |
| 16 | hr | No | No | No | No | No | No |
| 20 | hr | No | No | No | No | No | No |

### Example 9

The same procedure as reported for Example 7 was followed with the only exception being that D. vulgaris was replaced with D. sulfuicans. For all containers with microstructured surfaces (tCOC and Lexan) fluorescence signals from both the H₂S probe (AzMC) and the enzyme substrate (FDA or CFDA) were detected after 8 hours of incubation. However, the corresponding fluorescence signals were not detected in the anaerobic tubes (Comparative Example 7) after 20 hours of incubation. The results are summarized in Table 17 for the micro structured container and in Table 18 for the corresponding anaerobic tube (Comparative Example 7).

TABLE 17
Time to detect D. desulfuricans using both an H₂S Probe and an
Enzyme Substrate in a Container with a Microstructured Surface

| | Detection of Individual Fluorescence Signals Using | | | |
|---|---|---|---|---|
| | Both an H₂S Probe and Enzyme Substrate | | | |
| | AzMC + FDA | | AzMC + CFDA | |
| | Signal from | Signal from | Signal from | Signal from |
| Time | AzMC | FDA | AzMC | CFDA |
| 3 hr | No | No | No | No |
| 6 hr | No | No | No | No |
| 8 hr | Yes | Yes | Yes | Yes |
| 12 hr | Yes | Yes | Yes | Yes |
| 16 hr | Yes | Yes | Yes | Yes |
| 20 hr | Yes | Yes | Yes | Yes |

TABLE 18
Time to detect D. desulfuricans using both an H₂S Probe and an
Enzyme Substrate in an Anaerobic Tube (Comparative Example 7)

| | Detection of Individual Fluorescence Signals Using | | | |
|---|---|---|---|---|
| | Both an H₂S Probe and Enzyme Substrate | | | |
| | AzMC + FDA | | AzMC + CFDA | |
| | Signal from | Signal from | Signal from | Signal from |
| Time | AzMC | FDA | AzMC | CFDA |
| 3 hr | No | No | No | No |
| 6 hr | No | No | No | No |
| 8 hr | No | No | No | No |
| 12 hr | No | No | No | No |
| 16 hr | No | No | No | No |
| 20 hr | No | No | No | No |

Various features and aspects of the present disclosure are set forth in the following claims.

1. A method of detecting an analyte of interest comprising:
providing a container adapted to receive a sample, the container comprising a microstructured surface;
positioning the sample in the container;
adding an H₂S probe and an enzyme substrate to the container;
centrifuging the container toward the microstructured surface to form a sediment and a supernatant of the sample;
inverting the container, after centrifuging the container, to remove at least a portion of the supernatant of the sample from being in contact with the microstructured surface, such that a concentrate of the sample is retained in the microstructured surface, the concentrate comprising the sediment; and
interrogating the concentrate in the microstructured surface for the analyte of interest.

2. A method of detecting an analyte of interest comprising:
providing a container adapted to receive a sample, the container having an H₂S probe and an enzyme substrate, wherein the container comprises a microstructured surface;
positioning the sample in the container;
centrifuging the container toward the microstructured surface to form a sediment and a supernatant of the sample;
inverting the container, after centrifuging the container, to remove at least a portion of the supernatant from being in contact with the microstructured surface, such that a concentrate of the sample is retained in the microstructured surface, the concentrate comprising the sediment; and
interrogating the concentrate in the microstructured surface for the analyte of interest.

3. The method of claim 1, further comprises flushing the container with an inert gas before positioning the sample.

4. The method of, further comprises pressurizing the container.

5. The method of claim 1, wherein the microstructured surface forms at least a portion of an inner surface of the container.

6. The method of claim 1, wherein at least a portion of the container proximate the microstructured surface is substantially transparent to facilitate interrogating the concentrate from an exterior of the container.

7. The method of claim 1, wherein the microstructured surface comprises a plurality of microstructured recesses, each recess having a base, and wherein each base is substantially transparent.

8. The method of claim 7, wherein at least one of the plurality of microstructured recesses includes a sidewall, and wherein the sidewall is substantially non-transparent.

9. The method of claim 7, wherein each of the plurality of recesses contains a volume of no greater than 1 microliter.

10. The method of claim 7, wherein the microstructured surface includes a recess density of at least about 100 recesses per square centimeter.

11. The method of claim 1, wherein the container comprises an open end configured to receive a sample and a closed end, wherein the microstructured surface is formed in a first side of the closed end that is positioned to face the open end during centrifugation, wherein the closed end further comprises a second side opposite the first side.

12. The method of claim 11, wherein at least a portion of the closed end proximate the microstructured surface is substantially transparent.

13. The method of claim 11, wherein the container further comprises a cap to seal the open end.

14. The method of claim 11, wherein the container further comprises a septum between the cap and the open end.

15. An article comprising:
a container adapted to receive a sample, the container comprising an open end configured to receive a sample and a closed end, the closed end comprising:
a first side comprising a microstructured surface, the first side facing an interior of the container, and
a second side opposite the first side and facing outside of the container, wherein at least a portion of the container is substantially transparent such that the microstructured surface is visible from the second side;
a probe and an enzyme substrate disposed in the container.
